(19) United States
(12) Patent Application Publication
(10) Pub. No.: US 2025/0121134 A1
(43) Pub. Date: Apr. 17, 2025

(54) Hemodynamic Sensor Systems for Predicting and Diagnosing Endotypes of Hypotension

(71) Applicant: BD Switzerland Sàrl, Eysins (CH)

(72) Inventors: Xianfu Liu, Irvine, CA (US); Zhongping Jian, Irvine, CA (US); Feras Al Hatib, Irvine, CA (US); Jacobus Jozef Gerardus Maria Settels, De Hoef (NL); Brennan Michael Schneider, Irvine, CA (US)

(21) Appl. No.: 18/913,714

(22) Filed: Oct. 11, 2024

Related U.S. Application Data
(60) Provisional application No. 63/589,939, filed on Oct. 12, 2023.
(60) Provisional application No. 63/621,900, filed on Jan. 17, 2024.

(51) Int. Cl. A61M 5/172; G16H 20/17; G16H 40/67

(52) U.S. Cl. CPC A61M 5/1723; G16H 20/17; G16H 40/67; A61M 2205/18; A61M 2205/502; A61M 2230/30

(57) ABSTRACT

A system for determining an endotype of hypotension of a patient can include a hemodynamic sensor and a converter. The system can receive, from the hemodynamic sensor, an analog hemodynamic sensor signal from the patient. The system can convert, using the converter, the analog hemodynamic sensor signal to an arterial pressure signal waveform and extract from the arterial pressure signal waveform a plurality of heart health parameters. Using a deep learning model, the system can encode the plurality of heart health parameters into one or more latent space heart health parameters. The system can generate a location in latent space of the arterial pressure signal waveform and determine a relative location of the arterial pressure signal waveform in latent space. Based on the relative location, the system can determine the endotype of hypotension of the patient and display an alert indicating the endotype of hypotension.

### CROSS-REFERENCE TO RELATED APPLICATIONS

This application claims priority to U.S. Prov. Patent App. No. 63/589,939, filed Oct. 12, 2023, and U.S. Prov. Patent App. No. 63/621,900, filed Jan. 17, 2024, the disclosures of which are hereby incorporated herein by reference in their entireties.

### FIELD

The present disclosure relates generally to hemodynamic monitoring, including to determining an endotype of hypotension in a patient (e.g., human or veterinary subject) using monitored hemodynamic data.

### BACKGROUND

Monitoring hemodynamic variables of a patient allows for improved patient care. The hemodynamic variables can include heart health parameters, such as cardiac output. Monitoring such heart health parameters can allow a system to make diagnoses of an endotype of hypotension and provide interventions in hypotensive or potentially hypotensive patients. Systems and methods described herein provide potentially life-saving solutions in the space.

### SUMMARY

In one aspect, a system for determining an endotype of hypotension of a patient (e.g., human or veterinary subject) includes a hemodynamic sensor and a converter. The system can receive, from the hemodynamic sensor, an analog hemodynamic sensor signal from the patient. The system can convert, using the converter, the analog hemodynamic sensor signal to an arterial pressure signal waveform and extract from the arterial pressure signal waveform a plurality of heart health parameters. Using a fully connected deep learning model, the system can encode the plurality of heart health parameters into one or more latent space heart health parameters. The system can generate a location in latent space of the arterial pressure signal waveform and determine a relative location of the arterial pressure signal waveform in latent space. Based on the relative location, the system can determine the endotype of hypotension of the patient and display an alert indicating the endotype of hypotension.

In another aspect, a hemodynamic sensor system can determine and display an endotype of hypotension of a patient. The system can include a hemodynamic sensor that produces an analog hemodynamic sensor signal representative of an arterial pressure signal waveform of the patient, an analog-to-digital converter that converts the analog hemodynamic sensor signal to the arterial pressure signal waveform, a non-transitory memory having executable instructions stored thereon, and an electronic hardware processor in communication with the non-transitory memory. The system can receive, from the hemodynamic sensor, the analog hemodynamic sensor signal from the patient, and it can convert, using the analog-to-digital converter, the analog hemodynamic sensor signal to the arterial pressure signal waveform. The system can determine, based on the arterial pressure signal waveform, the endotype of hypotension of the patient and generate, based on the determined endotype of hypotension of the patient, data for displaying an alert indicating the endotype of hypotension of the patient.

In another aspect, the system can extract from the arterial pressure signal waveform a plurality of heart health parameters and encode, using a fully connected deep learning model, the plurality of heart health parameters into one or more latent space heart health parameters. In another aspect, the system can generate a location in latent space of the arterial pressure signal waveform using the one or more latent space heart health parameters and determine, based on the location of the arterial pressure signal waveform in latent space, a relative location of the arterial pressure signal waveform in latent space.

In another aspect, the system can determine a set of clusters from reference arterial pressure signal waveforms and determine, based on the set of clusters, a cluster associated with the arterial pressure signal waveform in latent space. Based on the cluster of the arterial pressure signal waveform, the system can determine the endotype of hypotension of the patient. The alert can further indicate at least one of the heart health parameters.

In another aspect, a hemodynamic sensor system can receive, from the hemodynamic sensor, an analog hemodynamic sensor signal from the patient and convert, using the analog-to-digital converter, the analog hemodynamic sensor signal to the arterial pressure signal waveform. The system can extract from the arterial pressure signal waveform a plurality of heart health parameters and encode, using a fully connected deep learning model, the plurality of heart health parameters into one or more latent space heart health parameters. The system can generate a location in latent space of the arterial pressure signal waveform using the one or more latent space heart health parameters. The system can determine, based on the location of the arterial pressure signal waveform in latent space, a relative location of the arterial pressure signal waveform in latent space. The system can obtain a plurality of reference arterial pressure signal waveforms from a plurality of patients and extract from the plurality of reference arterial pressure signal waveforms a plurality of reference sets of heart health parameters. The system can combine each of the plurality of reference sets of heart health parameters into a plurality of corresponding one or more reference latent space heart health parameters and generate reference locations in latent space of each of the plurality of reference arterial pressure signal waveforms based on the plurality of one or more reference latent space heart health parameters of each of the plurality of reference sets of heart health. The system can determine a clustering evaluation metric of the reference locations in latent space. The clustering evaluation metric system can indicate a goodness of clustering of the reference locations. Based on the clustering evaluation metric, the system can determine a best number of clusters associated with the reference locations. The system can associate, based on the best number of clusters, each of the reference arterial pressure signal waveforms to a corresponding cluster in latent space to determine a set of clusters and determine, based on the set of clusters, a cluster associated with the arterial pressure signal waveform in latent space. Based on the cluster of the arterial pressure signal waveform, the system can determine the endotype of hypotension of the patient and generate, based on the determined endotype of hypotension of the patient, data for displaying an alert indicating the endotype of hypotension of the patient.

In another aspect, the system can decode, using the fully connected deep learning model, the one or more latent space heart health parameters into the plurality of heart health parameters in original space. The system can determine, based on the decoded plurality of heart health parameters in original space, reference locations in original space of each of the plurality of reference arterial pressure signal waveforms and validate the determined best number of clusters associated with the reference locations by comparing the determined reference locations in latent space with the determined reference locations in original space.

In another aspect, the plurality of heart health parameters can include one or more of a first heart health parameter including a stroke volume index (SVI), a second heart health parameter including a heart rate (HR), a third heart health parameter including a cardiac index (CI), a fourth heart health parameter including a systemic vascular resistance index (SVRI), and/or a fifth heart health parameter including a stroke volume variation (SVV). The system can generate, based on the determined endotype of hypotension of the patient, data for displaying at least one of the first, second, third, fourth, or fifth heart health parameters.

In another aspect, the clustering evaluation metric includes at least one of a silhouette metric, a Calinski-Harabasz index, a Davies-Bouldin Index, and/or a Gaussian Mixture Model. The fully connected deep learning model includes an autoencoder configured to automatically convert the plurality of heart health parameters into the one or more latent space heart health parameters. The relative location of the arterial pressure signal waveform in latent space includes the cluster associated with the arterial pressure signal waveform in latent space.

In another aspect, the endotype of hypotension includes at least one of a vasodilation endotype, a myocardial depression endotype, a bradycardia endotype, and/or a hypovolemia endotype. The best number of clusters associated with the reference locations may be exactly four clusters.

In another aspect, a hemodynamic sensor system can receive, from a hemodynamic sensor, an analog hemodynamic sensor signal from the patient and convert, using an analog-to-digital converter, an analog hemodynamic sensor signal to the arterial pressure signal waveform. The system can extract from the arterial pressure signal waveform a plurality of heart health parameters and encode, using a fully connected deep learning model, the plurality of heart health parameters into one or more latent space heart health parameters. The system can generate a location in latent space of the arterial pressure signal waveform using the one or more latent space heart health parameters and determine, based on the location of the arterial pressure signal waveform in latent space, a relative location of the arterial pressure signal waveform in latent space. The system can determine a set of clusters from reference arterial pressure signal waveforms and determine, based on the set of clusters, a cluster associated with the arterial pressure signal waveform in latent space. Based on the cluster of the arterial pressure signal waveform, the system can determine the endotype of hypotension of the patient and generate, based on the determined endotype of hypotension of the patient, data for displaying an alert indicating the endotype of hypotension of the patient.

In another aspect, the system can decode, using the fully connected deep learning model, the one or more latent space heart health parameters into the plurality of heart health parameters in original space. The system can determine, based on the decoded plurality of heart health parameters in original space, reference locations in original space of each of the plurality of reference arterial pressure signal waveforms and validate the determined cluster associated with the arterial pressure signal waveform in latent space by comparing the determined reference locations in latent space with the determined reference locations in original space.

In another aspect, the plurality of heart health parameters includes one or more of a first heart health parameter including a stroke volume index (SVI), a second heart health parameter including a heart rate (HR), a third heart health parameter including a cardiac index (CI), a fourth heart health parameter including a systemic vascular resistance index (SVRI), and/or a fifth heart health parameter including a stroke volume variation (SVV). The system can generate, based on the determined endotype of hypotension of the patient, data for displaying at least one of the first, second, third, fourth, or fifth heart health parameters.

In another aspect, the system can determine a clustering evaluation metric of the reference arterial pressure signal waveforms, the clustering evaluation metric configured to indicate a goodness of clustering of the reference locations and, based on the clustering evaluation metric, determine a best number of clusters associated with the reference locations.

In another aspect, the clustering evaluation metric includes at least one of a silhouette metric, a Calinski-Harabasz index, a Davies-Bouldin Index, and/or a Gaussian Mixture Model. The best number of clusters associated with the reference locations may be exactly four clusters. The fully connected deep learning model includes an autoencoder configured to automatically convert the plurality of heart health parameters into the one or more latent space heart health parameters.

In another aspect, the relative location of the arterial pressure signal waveform in latent space includes the cluster associated with the arterial pressure signal waveform in latent space. The endotype of hypotension includes at least one of a vasodilation endotype, a myocardial depression endotype, a bradycardia endotype, and/or a hypovolemia endotype.

In another aspect, a hemodynamic sensor system can receive, from the hemodynamic sensor, a plurality of analog hemodynamic sensor signals from a plurality of reference patients and convert, using the analog-to-digital converter, a plurality of reference analog hemodynamic sensor signals to a corresponding plurality of reference arterial pressure signal waveforms. The system can extract from the plurality of reference arterial pressure signal waveforms corresponding sets of reference heart health parameters and encode, using the fully connected deep learning model, the plurality of reference sets of heart health parameters into a plurality of corresponding one or more reference latent space heart health parameters. The system can generate reference locations in latent space of each of the plurality of reference arterial pressure signal waveforms based on the plurality of one or more reference latent space heart health parameters of each of the plurality of reference sets of heart health. The system can determine a clustering evaluation metric of the reference locations in latent space, the clustering evaluation metric configured to indicate a goodness of clustering of the reference locations and, based on the clustering evaluation metric, determine a best number of clusters associated with the reference locations. The system can associate, based on the best number of clusters, each of the reference arterial pressure signal waveforms to a corresponding cluster in latent space to determine a set of clusters and determine, based on the set of clusters and on the reference locations in latent space of each of the plurality of reference arterial pressure signal waveforms, a cluster associated with each of the reference arterial pressure signal waveform in latent space. The system can determine, based on the cluster associated with each of the reference arterial pressure signal waveforms, the endotype of hypotension of each of the reference patients and decode, using the fully connected deep learning model, the plurality of one or more reference latent space heart health parameters into the corresponding plurality of reference sets of heart health parameters. The system can generate a trained model for determining the endotype of hypotension of the patient.

In another aspect, each of the sets of the plurality of reference heart health parameters includes one or more of a first heart health parameter including a stroke volume index (SVI), a second heart health parameter including a heart rate (HR), a third heart health parameter including a cardiac index (CI), a fourth heart health parameter including a systemic vascular resistance index (SVRI), and/or a fifth heart health parameter including a stroke volume variation (SVV). The system can generate, based on the determined endotype of hypotension of the patient, data for displaying at least one of the first, second, third, fourth, or fifth heart health parameters.

In another aspect, the clustering evaluation metric includes at least one of a silhouette metric, a Calinski-Harabasz index, a Davies-Bouldin Index, and/or a Gaussian Mixture Model. The best number of clusters associated with the reference locations is exactly four clusters. The fully connected deep learning model includes an autoencoder configured to automatically convert the plurality of heart health parameters into the one or more latent space heart health parameters. In another aspect, the endotype of hypotension includes at least one of a vasodilation endotype, a myocardial depression endotype, a bradycardia endotype, and/or a hypovolemia endotype.

### BRIEF DESCRIPTION OF THE DRAWINGS

FIG. 1 is a block diagram of a hemodynamic sensing system that can determine an endotype of hypotension for a patient based on hemodynamic signal data, according to some embodiments.

FIG. 2 is a graph illustrating an example arterial pressure signal waveform.

FIG. 3 is a perspective view of an example hemodynamic sensor that can be coupled (e.g., attached) to the patient for sensing hemodynamic data representative of arterial pressure of the patient, according to some embodiments.

FIG. 4 is a perspective view of an example hemodynamic sensor for sensing hemodynamic data representative of arterial pressure of the patient, according to some embodiments.

FIG. 5A is a block diagram illustrating an example autoencoder model of an endotype analysis system, according to some embodiments.

FIG. 5B shows an example autoencoder model using fully connected encoding and decoding layers, according to some embodiments.

FIG. 5C shows an example autoencoder model using convoluted encoding and decoding layers.

FIG. 6A shows a graph of example results of calculating a goodness of clustering across a potential number of clusters in latent space, ranging from 2 to 14 clusters.

FIG. 6B shows a graph of an example evaluation of a goodness of clustering of a set of locations in original space using a clustering evaluation metric.

FIG. 6C shows a graph of example results from an arterial pressure signal waveform cluster determination.

FIG. 6D shows a plurality of box-and-whisker plots of example results of a trained machine learning model that is configured to diagnose or determine an endotype of hypotension of a test patient.

FIGS. 6E-6J illustrate example user interfaces depicting endotype trends associated with a patient.

FIG. 7 shows an example of a method for determining an endotype of hypotension of a patient.

FIG. 8 shows an example of a method for generating a trained model for determining an endotype of hypotension of a patient.

FIG. 9 is a block diagram that illustrates a computer system upon which various implementations may be implemented.

### DETAILED DESCRIPTION

### Overview

Hypotension, or low blood pressure, is a physiological condition characterized by a blood pressure reading below an acceptable threshold level. An acceptable threshold level of blood pressure may be generally considered to be at least 90/60 mm Hg for most people (e.g., 90 mm Hg for a systolic pressure threshold, and 60 mm Hg for a diastolic pressure threshold level), below which may be considered hypotension for most people. The definition of hypotension can vary based on the individual circumstances, so some people may naturally have lower blood pressure without experiencing any hypotensive symptoms. Like its counterpart of hypertension, hypotension can also pose health risks and warrant serious medical attention. Blood pressure is a vital component of cardiovascular health, and deviations from an acceptable range can impact organ perfusion and overall well-being.

Traditionally, the diagnosis of hypotension involves measuring blood pressure using a sphygmomanometer. A systolic pressure below a systolic pressure threshold level (e.g., about 90 mm Hg) and/or a diastolic pressure below a diastolic pressure threshold level (e.g., about 60 mm Hg) may be considered indicative of hypotension. However, this conventional approach might overlook underlying complexities that contribute to the condition. Identifying one or more endotypes of hypotension in a patient can be an important step in properly treating the particular needs of a hypotensive or potentially hypotensive patient. Endotypes represent distinct subtypes or mechanisms that lead to, or result in, hypotension in individual patients. An endotype of hypotension generally refers to a specific type of low blood pressure characterized by distinct physiological or molecular features.

The systems described herein can identify and diagnose various endotypes of hypotension. Each endotype may include diverse etiological factors that contribute to low blood pressure. These may include, for example, vasodilation, hypovolemia, myocardial depression, bradycardia, and/or other endotypes. The systems described herein can additionally or alternatively identify a number of proper categorizations of endotypes based on a large sample of hypotensive patients. Several embodiments of the invention are particularly advantageous because they include one, several or all of the following benefits: (i) reduce or prevent mistakes in diagnosing endotypes of hypotension, (ii) allow for diagnosis of endotypes of hypotension in real-time, including during emergency situations, (iii) use a combination of fully connected layers and unsupervised learning algorithms to overcome limitations in the function of computers in diagnosing endotypes of hypotension, and/or (iv) generate real-time output to local and/or remote computing devices based on updated data in real-time, and/or validate the effects of encoding data by decoding the data and comparing it with the unencoded data.

A hemodynamic sensing or monitoring system can be used to diagnose an endotype of a patient in real time. Such systems may be more accurate and/or rapid than human diagnoses. Properly diagnosing (or, in some cases, predicting) an endotype of hypotension can result in better (e.g., more effective or more rapid) treatment. For example, vasodilation may be characterized by an abnormal widening or dilation of blood vessels. Accordingly, using vasopressors to constrict blood vessels may be appropriate. By contrast, hypovolemia can result from a significant loss of fluid (e.g., blood) from the body. Treatment of hypovolemia may include providing the patient with intravenous fluid, such as saline or colloids.

The hemodynamic sensing system may obtain an analog arterial pressure signal. This may be an analog hemodynamic sensor signal (e.g., analog hemodynamic signal) that can be converted to a different form (e.g., digital form) of signal, such as an arterial pressure signal waveform. The hemodynamic sensing system can use machine learning to extract sets of parameters, such as heart health parameters, from the arterial pressure of the patient. As described herein, “heart health parameters” can have its plain and ordinary meaning and may generally refer to health parameters associated with cardiovascular health (e.g., vascular health, blood health, etc.) and need not be specific to the heart. The sets of input features can be used by the hemodynamic sensing system to determine one or more endotypes of hypotension of a patient while the patient is visiting an office of a primary care physician, while in an emergency care setting, and/or in any other patient care environment. In some embodiments, the hemodynamic sensing system can even be made available “over the counter” for use at home by the patient.

Depending on the severity of the particular endotype of hypotension detected by the hemodynamic sensing system, the hemodynamic sensing system can generate a signal or an alarm to medical workers and/or the patient to alert the medical workers and/or the patient that the patient requires attention (e.g., immediate emergency attention).

### Hemodynamic Sensing System

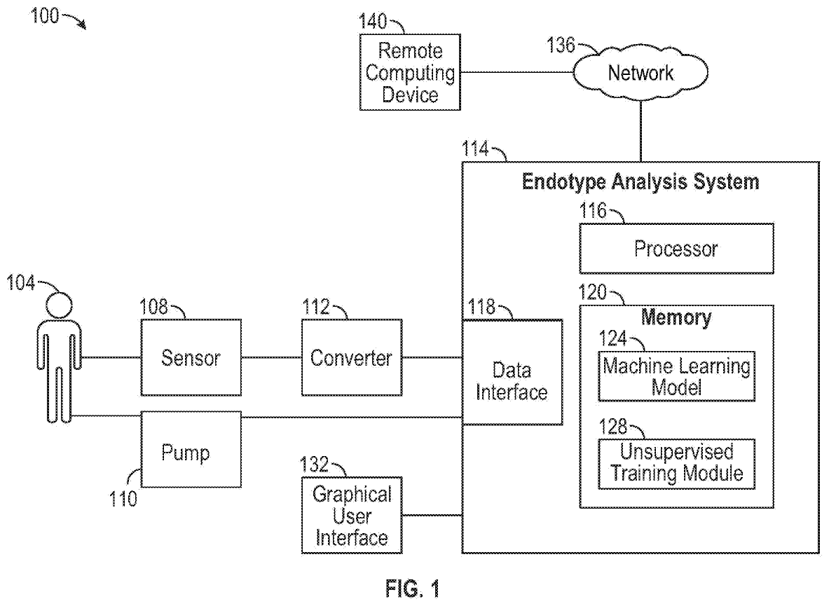

FIG. 1 is a block diagram of a hemodynamic sensing system 100 that can determine an endotype of hypotension for a patient based on hemodynamic signal data, according to some embodiments. As illustrated in FIG. 1, the hemodynamic sensing system 100 includes a hemodynamic sensor 108 coupled to a patient 104, a signal converter 112, an endotype analysis system 114, and/or a graphical user interface 132. Additionally or alternatively, the hemodynamic sensing system 100 can include a pump 110 coupled to the patient 104. In some embodiments, the hemodynamic sensing system 100 includes a remote computing device 140 connected via a network 136. The endotype analysis system 114 can include one or more processors 116, a hemodynamic data interface 118, and/or a memory 120. The memory 120 can include instructions (e.g., software instructions) stored thereon for implementing one or more steps described herein. Additionally or alternatively, the memory 120 can include a machine learning model 124 and/or an unsupervised training module 128. The hemodynamic sensing system 100 can be implemented within a patient care environment, such as an intensive care unit (ICU), an operating room (OR), and/or other patient care environment.

For example, in some embodiments, the hemodynamic sensing system 100 includes a hemodynamic sensor 108, a signal converter 112, a memory 120, and one or more processors 116. The one or more processors 116 are configured to execute instructions stored on the memory 120 to receive, from the hemodynamic sensor 108, an analog hemodynamic sensor signal from a patient. The one or more processors 116 can cause the hemodynamic sensing system 100 to convert, using the signal converter 112, the analog hemodynamic sensor signal to the arterial pressure signal waveform and determine, based on the arterial pressure signal waveform, an endotype of hypotension of the patient.

The one or more processors 116 can be one or more hardware and/or electronic processors. The processor(s) 116 can include one or more of a microprocessor, a controller, a digital signal processor (DSP), an application specific integrated circuit (ASIC), a field-programmable gate array (FPGA), or other equivalent discrete or integrated logic circuitry. In some embodiments, the one or more processors 116 can include one or more graphical processing units (GPUs). The one or more GPUs may be configured to conduct linear algebraic calculations on matrices. For example, the one or more GPUs may be used by the machine learning model 124 and/or the unsupervised training module 128 to perform the operations described below.

The memory 120 can include computer-readable storage media. In some examples, a computer-readable storage medium can include a non-transitory medium. The term “non-transitory” can indicate that the storage medium is not embodied in a carrier wave or a propagated signal. In certain examples, a non-transitory storage medium can store data that can, over time, change (e.g., in RAM or cache). The memory 120 can include volatile and non-volatile computer-readable memories. Examples of volatile memories can include random access memories (RAM), dynamic random access memories (DRAM), static random-access memories (SRAM), and other forms of volatile memories. Examples of non-volatile memories can include, e.g., magnetic hard discs, optical discs, flash memories, or forms of electrically programmable memories (EPROM) or electrically erasable and programmable (EEPROM) memories.

In some embodiments, the hemodynamic sensing system 100 can include or be in communication with a remote computing device 140 via the network 136. The remote computing device 140 can include a computing system such as a client terminal at a hospital or clinic. The remote computing device 140 may include a computer in the ICU or in the OR. Additionally or alternatively, the remote computing device 140 may include a mobile electronic device, such as a laptop, a smartphone, a health monitor, and/or other electronic device. In some embodiments, the remote computing device 140 may include a display interface. The remote computing device 140 may be configured to receive (e.g., via the network 136) and/or display data transmitted from the endotype analysis system 114, such as a determined endotype of hypotension, one or more heart health parameters described herein, imagery associated with the heart health of the patient 104, and/or other data. The network 136 can include a wireless and/or wired network connection.

### Hemodynamic Sensor

The hemodynamic sensor 108 can include one or more sensors coupled to (e.g., attached to, inserted within, etc.) the patient 104. The hemodynamic sensor 108 can obtain (e.g., receive, sense) a hemodynamic signal representative of an arterial pressure waveform of the patient 104 (see, e.g., the arterial pressure signal waveform 200). The sensed signal can be converted to data (e.g., digital data) by the signal converter 112.

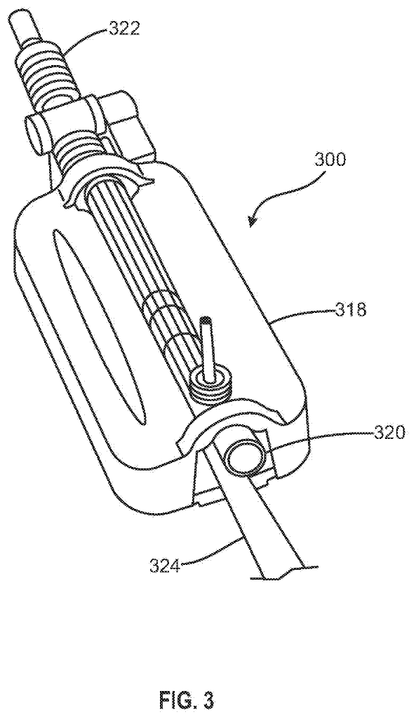

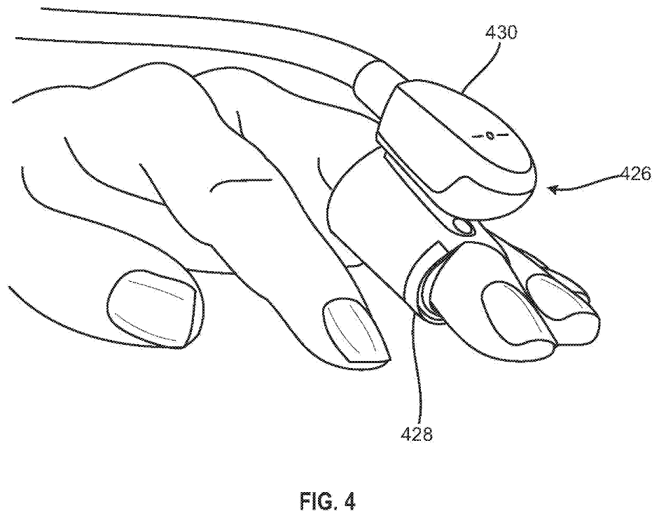

The hemodynamic sensor 108 can be inserted into the patient via a femoral arterial catheter inserted into a leg of the patient. Additionally or alternatively, the hemodynamic sensor 108 can include a minimally invasive hemodynamic sensor that can be attached to the patient via, e.g., a radial arterial catheter inserted into an arm of the patient (see, e.g., the hemodynamic sensor 300). For example, in some embodiments, the hemodynamic sensor 108 includes a non-invasive hemodynamic sensor that can be attached to the patient via one or more finger cuffs configured to sense data representative of arterial pressure of the patient. For example, the hemodynamic sensor 108 can include an inflatable finger cuff and a heart reference sensor (see, e.g., FIG. 3). In some embodiments, the hemodynamic sensor 108 does not include any invasive hemodynamic sensor (e.g., an in-line hemodynamic sensor, such as the hemodynamic sensor 300). In some embodiments, the hemodynamic sensor 108 includes a wireless (e.g., infrared) or wired connection to the signal converter 112.

The hemodynamic sensor 108 can take regular hemodynamic signal measurements of the patient 104. The measurements may take place about every 5 s, about every 10 s, about every 20 s, about every 30 s, about every 45 s, about every 1 min, about every 2 min, about every 5 min, about every 10 min, about every 15 min, about every 20 min, about every 30 min, about every 1 hour, any value therein, or fall within a range having endpoints therein. For example, in some embodiments the measurements are taken about every 15 minutes. The rate of measurement may be determined in part by the determined heart health of the patient, such as, for example, whether the patient 104 is currently in hypotension, how many endotypes of hypotension have been identified for the 104, whether one or more of the heart health parameters exceeds corresponding one or more thresholds, etc. The determined rate of measurement may be automatically determined or may be set by a user. While the hemodynamic sensor 108 can monitor the arterial pressure of the patient 104 over an extended period of time, the hemodynamic sensor 108 may only need to monitor the arterial pressure of patient 104 for a few minutes (e.g., 5 minutes) to generate enough data for the endotype analysis system 114 to determine an endotype of hypotension of the patient 104.

### Signal Converter

The signal converter 112 may include an analog-to-digital converter (ADC) and/or a digital-to-analog converter (DCA). The signal converter 112 may include a hardware and/or software converter. The signal converter 112 can transmit the converted data to the endotype analysis system 114. Example signal converters 112 are described below with reference to FIGS. 3 and 4.

### Endotype Analysis System

The endotype analysis system 114 can receive the converted data from the signal converter 112 via the hemodynamic data interface 118. In some embodiments, the signal converter 112 is configured to convert the data to a form (e.g., format) that can be read and/or accepted by the hemodynamic data interface 118. Once the converted data is obtained by the endotype analysis system 114, the processor 116 can execute instructions stored on the memory 120 to conduct analysis on the converted data.

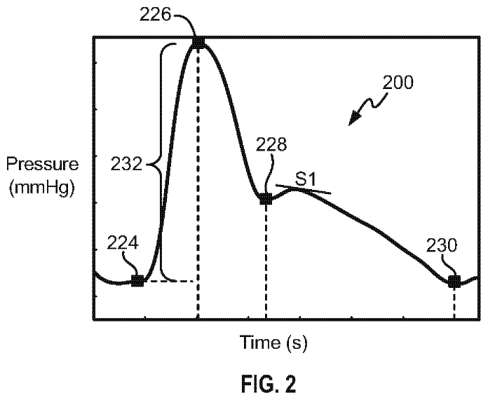

The converted data can comprise one or more health parameters, such as heart health parameters (see also FIG. 2). The heart health parameters may be highly predictive of potential (e.g., future) or actual (e.g., present) hypotension for the patient 104. These heart health parameters may be derived from the digital hemodynamic waveform data. The hemodynamic sensing system 100 can utilize some or all of the heart health parameters to diagnose one or more endotypes of hypotension and/or a hypotension probability index (hereinafter “HPI”) corresponding to the probability of a future hypotension event and/or an associated endotype therefor for the patient 104. For example, the endotype analysis system 114 can determine an HPI corresponding to a probability of the patient 104 developing one or more endotypes of hypotension. The probability may be used to perform one or more actions described herein, such as determining a therapy protocol for the patient and/or generating a command to cause an infusion pump to deliver therapy to the patient 104. For example, the endotype analysis system 114 may determine a particular endotype of hypotension based on a determination that the HPI is above a threshold HPI (e.g., 90, 95, 100, etc.). The endotype analysis system 114 may use the hearth health parameters to determine an endotype and/or determine the HPI by comparing the heart health parameters to a reference, such as a table of values (e.g., a lookup table), such as described herein.

As described herein, one or more of the heart health parameters may be transmitted to the graphical user interface (GUI) 132 for display. The graphical user interface 132 can alert a user (e.g., a healthcare professional or patient himself or herself) about the determined endotype and/or recommend an action item to address that particular endotype. Such an alert can help ensure that a timely warning of a potential emergency hypotension event is provided to the user. Moreover, by enabling the user to access the graphical user interface 132 showing or displaying the one or more heart health parameters identified as indicative of the present or future hypotension endotype, the graphical user interface 132 can provide detailed diagnostic information allowing the user to identify a most probable cause of the endotype of hypotension and/or best medical interventions for the prevention or treatment of that particular endotype.

### Machine Learning Model

With further reference to FIG. 1, the endotype analysis system 114 can be configured to identify one or more of the heart health parameters relevant to identification of an endotype of hypotension and/or to determine the endotype of hypotension itself. The heart health parameters can include health parameters that can be measured from the arterial pressure signal waveform and/or that may be useful in identifying (e.g., diagnosing) an endotype of hypotension. There are many potential heart health parameters that may be extracted from the arterial pressure signal waveform, but they can include, for example, cardiac output (CO), stroke volume (SV), stroke volume variation (SVV), diastolic pressure (DIA), pulse rate (PR), stroke volume index (SVI), systemic vascular resistance (SVR), mean arterial pressure (MAP), HPI, and/or others. Additionally or alternatively, the heart health parameters can include systemic vascular resistance index (SVRI), cardiac index (CI), and/or systolic pressure (SYS).

The endotype analysis system 114 may receive the converted data (e.g., the heart health parameters) from the signal converter 112 via the hemodynamic data interface 118. The endotype analysis system 114 may then transmit the heart health parameters to the machine learning model 124 and/or the unsupervised training module 128. The machine learning model 124 can be configured to receive the heart health parameters and encode them into different health parameters. Before encoding, the heart health parameters can be considered to be in “original space.” Original space can refer to a mathematical or analysis space of values before they are encoded and/or after they are decoded. Once encoded, the different health parameters may be heart health parameters in a different space (e.g., latent space). A “latent space” can refer to a mathematical or analytical space of values after being encoded or compressed (e.g., by an autoencoder described below) and/or before being decoded. Values in latent space may be more compact (e.g., lower dimension) than values in original space. Latent space values may not be directly observable. In some embodiments, the machine learning model 124 is not configured encode the heart health parameters into any other space than the latent space. Additionally or alternatively, the machine learning model 124 may be configured to decode latent space into original space and no higher-dimensional space. These different heart health parameters may be transmitted by the machine learning model 124 to the unsupervised training module 128.

In some embodiments, the machine learning model 124 can further decode the latent space heart health parameters back into corresponding heart health parameters. The machine learning model 124 can include a fully connected deep learning model, a convoluted deep learning model, and/or some other type of learning model. In some embodiments, one or more of these heart health parameters can be transmitted to the graphical user interface 132 for display to a healthcare professional.

The machine learning model 124 can include an autoencoder model (or “autoencoder”). The autoencoder model can receive raw or unencoded data (e.g., the heart health parameters) as input and output encoded (and/or decoded) parameters based on the heart health parameters. The autoencoder model can be a deep-learning-based model. The autoencoder model can be trained to convert the unencoded heart health parameters into encoded heart health parameters, as discussed in more detail below.

### Unsupervised Learning Module

The unsupervised training module 128 can receive the different heart health parameters to generate corresponding locations of the heart health parameters in the different space based on the encoding of the heart health parameters. The location may be in a latent space or other space, as described below. The unsupervised training module 128 can use one or more criteria or sets of criteria to determine how these heart health parameters should be located.

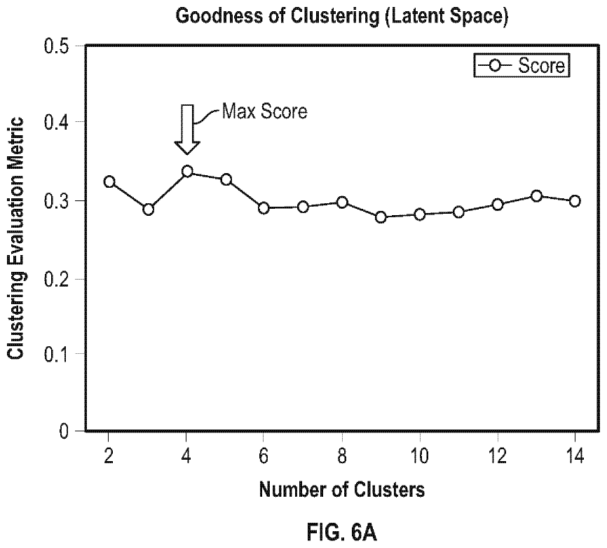

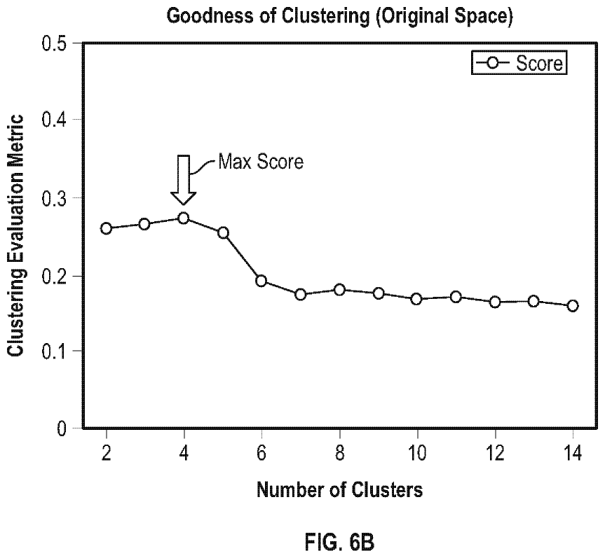

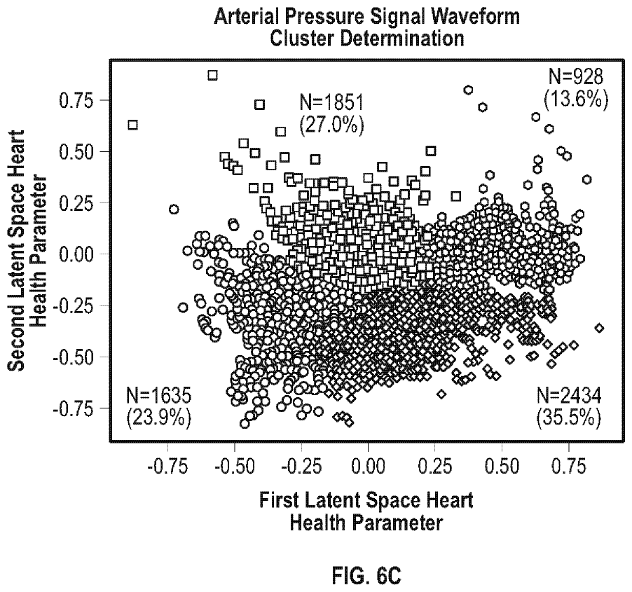

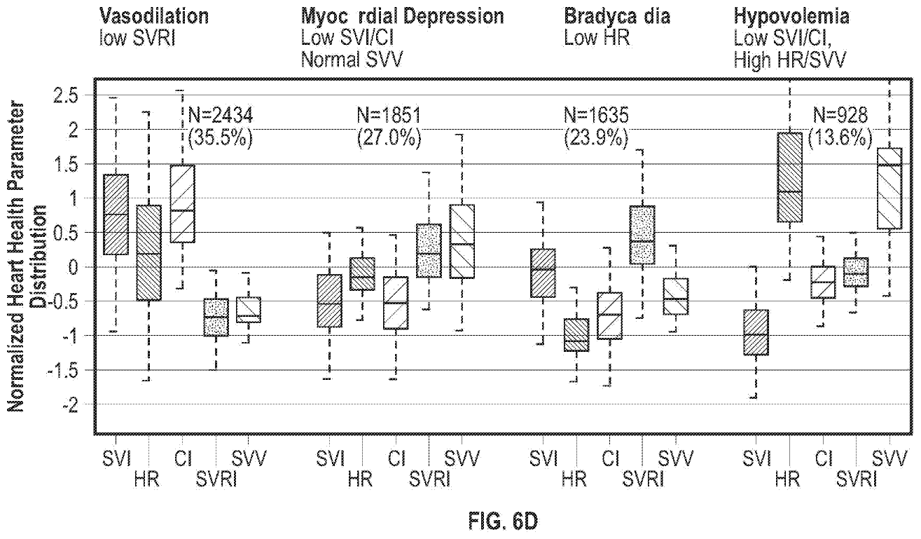

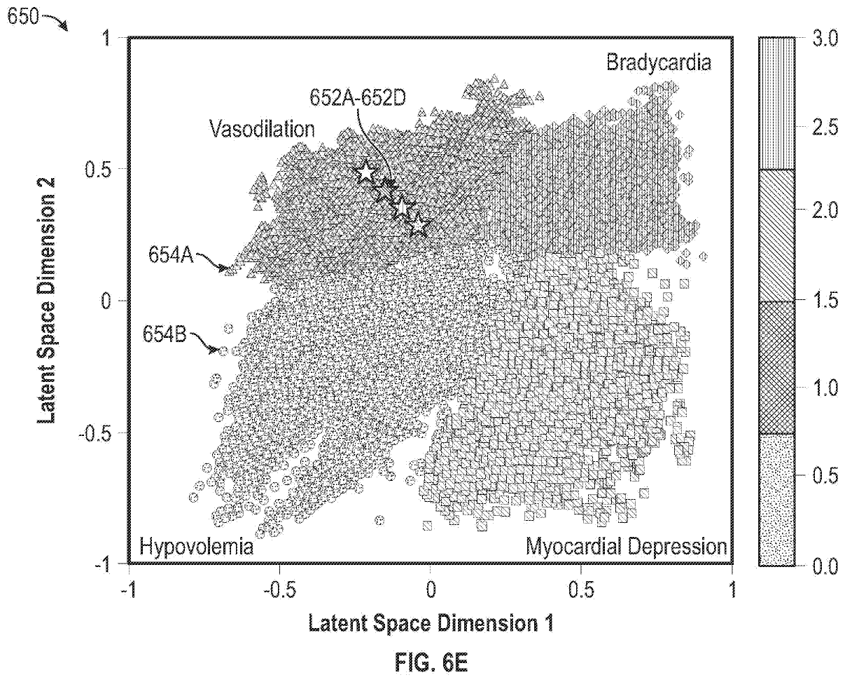

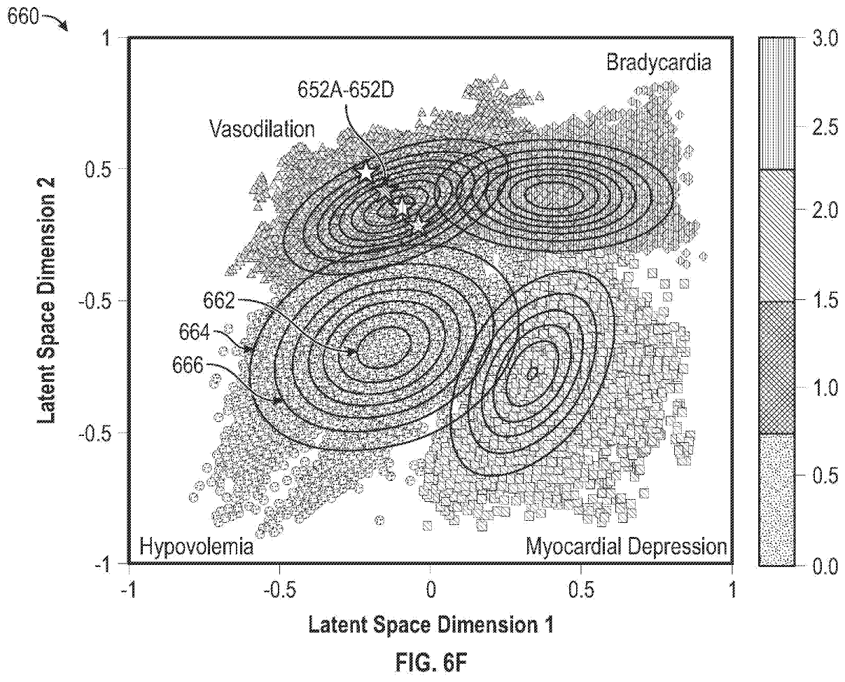

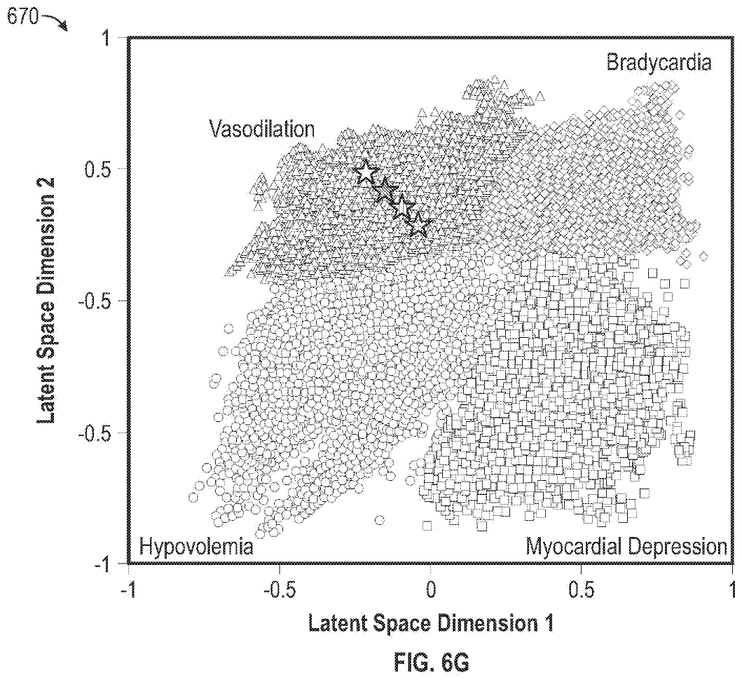

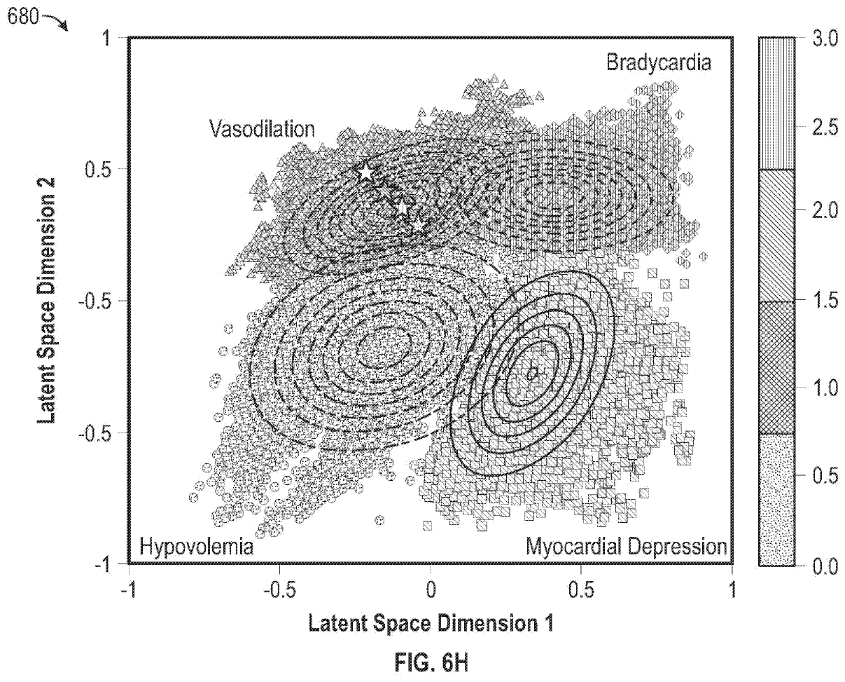

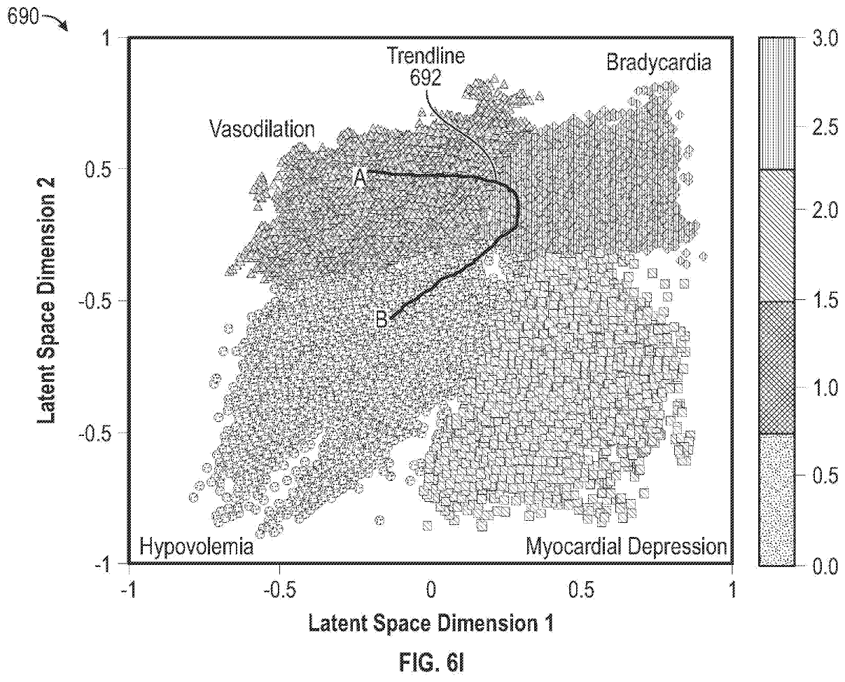

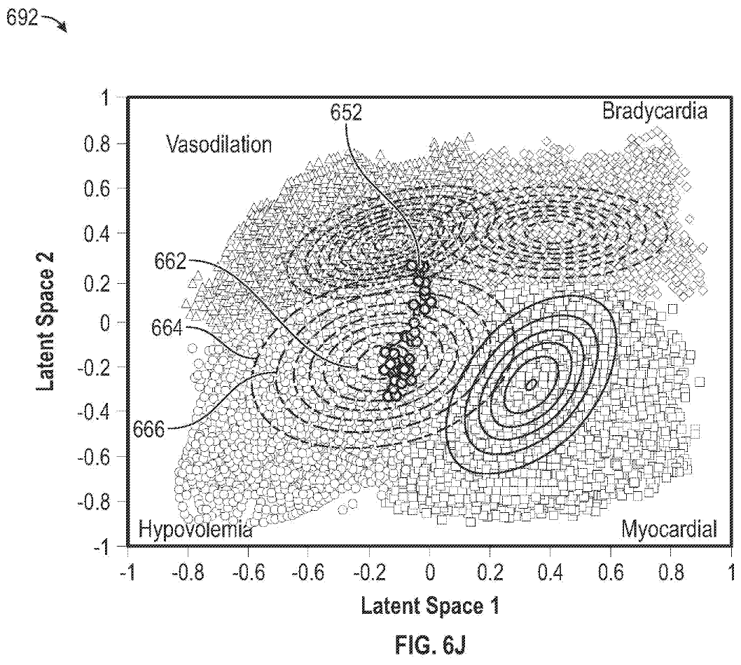

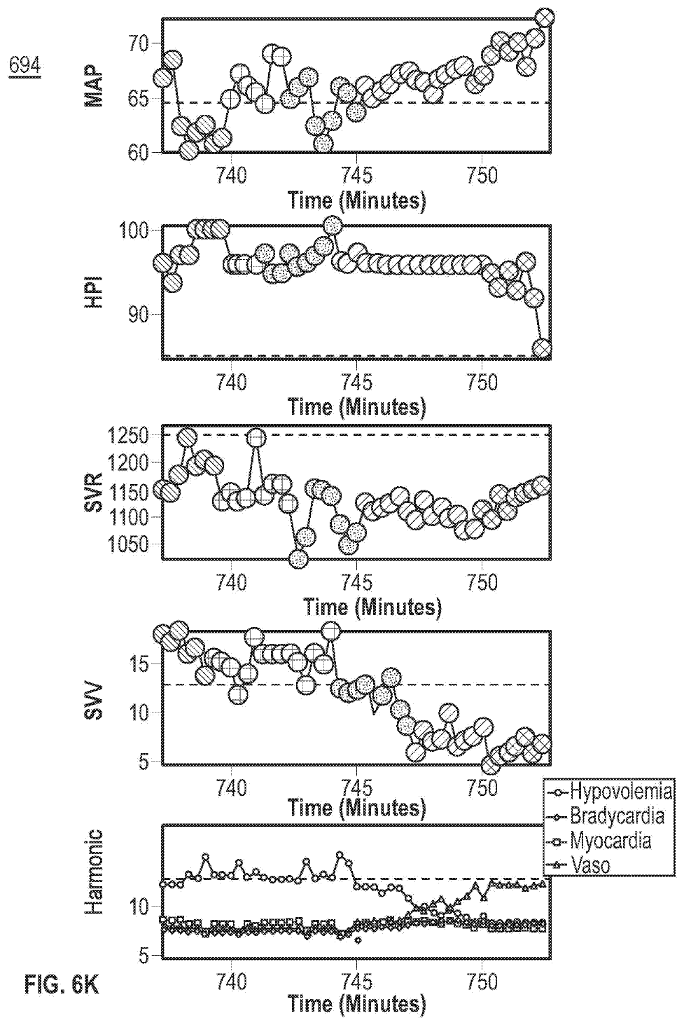

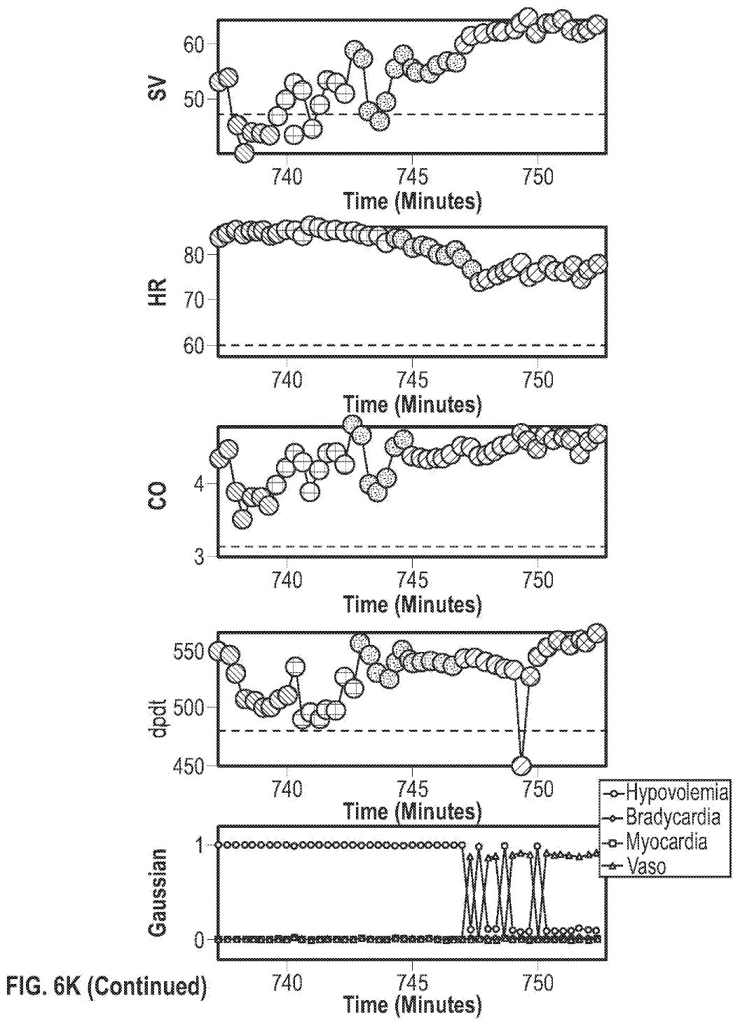

The unsupervised training module 128 can identify a number of clusters for the model and determine a location (e.g., which may be a reference location) in the different space of each location within a particular cluster. In some embodiments, additionally or alternatively the unsupervised training module 128 can identify a number of clusters for the model and determine a location in the original space. The clustering in the original space may be used to validate the clustering in the different (e.g., latent) space. In some embodiments, the unsupervised training module 128 is not constrained by a number of clusters. For example, in contrast to supervised training modules, the unsupervised training module 128 may be allowed to calculate any number of potential clusters without limitation. In some embodiments, the endotype analysis system 114 may compare one or more of the hearth health parameters with a reference to determine an endotype. The reference may include a range of values for that particular heart health parameter (e.g., see FIG. 6D). The endotype analysis system 114 can use a plurality of references for associated indices (e.g., SVI, HR, CI, SVRI, SVV, etc.) to determine an endotype and/or a probability that an endotype applies. The reference may include a table (e.g., lookup table). Additionally or alternatively, the reference may include one or more thresholds, such as a maximum threshold and/or a minimum threshold.

### Graphical User Interface

The unsupervised training module 128 can receive the different heart health parameters to generate corresponding locations of the heart health parameters in the different space based on the encoding of the heart health parameters. The location may be in a latent space or other space, as described below. The unsupervised training module 128 can use one or more criteria or sets of criteria to determine how these heart health parameters should be located.

The graphical user interface 132 can provide a user interface that includes one or more control elements to enable user interaction and/or input therein. User input may be transmitted to the endotype analysis system 114. The graphical user interface 132 may provide a sensory alarm based on a determined endotype and/or based on measured data from the arterial pressure signal waveform (e.g., from the one or more extracted heart health parameters). The sensory alarm can be configured to provide a warning to medical personnel based on whether the endotype of hypotension raises an emergency. The sensory alarm may additionally or alternatively include instructions for how to treat the determined endotype of hypotension, the level of urgency of treatment, and/or relevant heart health parameters that should be addressed based on the endotype of hypotension. For example, a hypovolemia endotype may require emergency delivery of saline. The sensory alarm 158 can be implemented as one or more of a visual alarm, an audible alarm, a haptic alarm, and/or other type of sensory alarm. For example, the sensory alarm can be invoked as any combination of flashing and/or colored graphics shown by the graphical user interface 132. Additionally or alternatively, the graphical user interface 132 may display the determined endotype of hypotension via graphical user interface 132, a warning sound such as a siren or repeated tone, and a haptic alarm configured to cause a hemodynamic monitor (not shown) to vibrate or otherwise deliver a physical impulse perceptible to a medical worker or other user. The signal for the haptic alarm may be transmitted wirelessly via the network 136.

The graphical user interface 132 can include a liquid crystal display (LCD), a light-emitting diode (LED) display, an organic light-emitting diode (OLED) display, or other display device suitable for providing information to users in graphical form. The graphical user interface 132 can include one or more touch-sensitive and/or presence sensitive elements, such as a touch-sensitive display screen. In some embodiments, user input can be received in the form of gesture input, such as touch gestures, scroll gestures, zoom gestures, or other gesture input. In some examples, the graphical user interface 132 can include one or more physical control elements, such as a physical buttons, keys, knobs, mouse, keyboard, or other physical control elements configured to receive user input to interact with components of the hemodynamic sensing system 100. However, in some embodiments the graphical user interface 132 does not allow for user selection (e.g., does not take user input) but instead provides only output to a user.

### Pump

With further reference to FIG. 1, the hemodynamic sensing system 100 can include a pump 110 that can provide a therapy to the patient 104. The endotype analysis system 114 can communicate with the pump 110 (e.g., via the data interface 118). The pump 110 can include an infusion pump. For example, the pump 110 can include a gravity infusion pump, a syringe infusion pump, an elastomeric infusion pump, a volumetric infusion pump, a patient-controlled analgesia (PCA) pump, an enteral infusion pump, an insulin pump, an ambulatory infusion pump, and/or any other kind of infusion pump.

A gravity infusion pump can use gravity to deliver fluids into the an intravenous (IV) line and ultimately into the patient's body. The infusion container may be located above the patient 104 for gravity to have its effect. A syringe infusion pump may be built in or directly connected to the pump system. A syringe infusion pump can provide small volume infusions and/or offer increased control over the fluid delivery.

An elastomeric infusion pump can be a single-use pump that functions without an external power source. Such a pump 110 can include one or more elastomeric balloons filled with fluid, which can create a positive pressure to push the fluid out along the IV line. In some embodiments, the pump 110 can allow the patient 104 to at least partially control the administration of therapy, such as in PCA pumps.

The pump 110 can receive instructions from the endotype analysis system 114, such as via the data interface 118. The endotype analysis system 114 can generate a control signal to instruct the pump 110 to deliver therapy to the patient 104, such as intravenous therapy using an intravenous therapeutic agent. The therapy provided may be based on an endotype determined by the endotype analysis system 114. Additionally or alternatively, the therapy may be based on a therapeutic protocol, such as one determined by the endotype analysis system 114. The therapeutic protocol may include instructions relating to a type (or types) of therapeutic agent(s), an amount of the therapeutic agent(s), a length of time associated with delivery of the therapeutic agent(s), and/or any other therapy described herein.

The therapy can include intravenous infusion of one or more fluids in order to expand plasma volume, which can help increase blood pressure. The type of fluid(s) used (e.g., crystalloid and/or colloid) can be based on the patient's condition (e.g., endotype and/or likelihood of developing that endotype) and the desired effect. The fluid therapy can correct a volume deficit and/or provide additional oxygen. Additionally or alternatively, the therapy may include one or more vasopressors, which can include medications used to constrict blood vessels. This can increase a vascular resistance and blood pressure. Vasopressors can stimulate receptors in the cardiovascular system to induce vasoconstriction. Additionally or alternatively, the pump 110 can deliver inotropes: These drugs enhance the contractility of the heart muscle, improving cardiac output. Inotropes can improve myocardial contractility and/or heart rate, which in turn may support better blood pressure control. In some embodiments, the therapy can include fluid resuscitation, which can increase blood volume and/or improve tissue perfusion. The therapeutic agents may be initially titrated using a lower dose, possibly with a ramped up dose over time, to reduce a likelihood of adverse effects.

### Arterial Pressure Signal Waveform

FIG. 2 is a graph illustrating an example arterial pressure signal waveform 200. The arterial pressure signal waveform 200 can include a plurality of attributes each indicative of one or more aspects of blood flow, cardiac output, and/or other aspects of heart health parameters described herein. The arterial pressure signal waveform 200 can correspond to a single heartbeat of the patient 104. The hemodynamic sensor 108 can be configured to detect new arterial pressure signal waveforms 200 for a plurality of heartbeats in a particular timeframe. For example, the hemodynamic sensor 108 may be able to generate a new arterial pressure signal waveform 200 for each heartbeat.

The arterial pressure signal waveform 200 is an example waveform that corresponds to hemodynamic data sensed by the hemodynamic sensor 108 and converted by the signal converter 112. Arterial pressure signal waveform 200 (represented via digital hemodynamic data) can include various indicia indicative of heart health for the patient 104. Various heart health parameters can be extracted by the endotype analysis system 114, as discussed herein. Prior to extracting the heart health parameters, a beat detector can identify a start and an end of each heartbeat corresponding to each waveform. The beat detector can be a hardware detector (e.g., a resistance detector, an inductance detector, optical detector, etc.) or a software detector, such as a software beat detector. The beat detector may identify the start of the heartbeat based on a maximum arterial pressure, a minimum arterial pressure, a maximum or minimum rate of change in the arterial pressure, and/or a second derivative in the arterial pressure with respect to time in the arterial pressure. Based on the heartbeat identification within the arterial pressure signal waveform 200, various health parameters of heart health can be extracted from the waveform on an on-going (e.g., real-time), beat-to-beat basis. In some embodiments, the endotype analysis system 114 can obtain any necessary input data from a single arterial pressure signal waveform 200 without needing to rely on a plurality of such arterial pressure signal waveforms.

The start indicator 224 of the arterial pressure signal waveform 200 corresponds to the start of a heartbeat. The systolic maximum indicator 226 of the arterial pressure signal waveform 200 corresponds to a maximum systolic pressure, marking an end of systolic rise. The notch indicator 228 of the arterial pressure signal waveform 200 corresponds to a presence and corresponding pressure of a dicrotic notch, marking an end of systolic decay. The diastolic minimum indicator 230 of the arterial pressure signal waveform 200 corresponds to a minimum diastolic pressure of the heartbeat of the patient 104. Further, arterial pressure gradients, or pressure differences between points of the arterial pressure signal waveform 200, can be used to identify the heart health parameters. For instance, a pulmonary pulse pressure 232 represents the difference between minimum diastolic pressure (the diastolic minimum indicator 230) and maximum systolic pressure (the systolic maximum indicator 226). As shown, slope S2 is a slope of the arterial pressure signal waveform 200, which may also be useful in determining the heart health parameters. The slope S2 is depicted at one location but is representative of multiple slopes that may be determined at multiple locations along arterial pressure signal waveform 200. For instance, a maximum and/or a minimum time derivative of the arterial pressure signal waveform 200 may be used to calculate the heart health parameters.

Additional indicators of the arterial pressure signal waveform 200 can be useful for calculating heart health parameters. For example, an interval between the systolic maximum indicator 226 and the notch indicator 228 can be extracted from the arterial pressure signal waveform 200. Additionally or alternatively, an interval between the start indicator 224 and the diastolic minimum indicator 230 can be extracted from the arterial pressure signal waveform 200. The endotype analysis system 114 may use these and/or other indicators from the arterial pressure signal waveform 200 to identify additional heart health parameters from the arterial pressure signal waveform 200. For example, a systolic rise (e.g., between the start indicator 224 and the systolic maximum indicator 226), a systolic decay (e.g., between the systolic maximum indicator 226 and the notch indicator 228), a systolic phase (e.g., between the start indicator 224 and the notch indicator 228), a diastolic phase (e.g., between the notch indicator 228 and the diastolic minimum indicator 230), and/or a heartbeat interval (between successive start indicators 224) can be determined by the endotype analysis system 114. Such indicia may include the mean arterial pressure during one of the above-referenced intervals. The area under the curve of arterial pressure signal waveform 200 and the standard deviations of the arterial pressure signal waveform 200 determined for the above-referenced intervals can also serve as heart health parameters and/or inputs for calculating the same.

### Example Hemodynamic Sensors

FIG. 3 is a perspective view of an example hemodynamic sensor 300 that can be coupled (e.g., attached) to the patient 104 for sensing hemodynamic data representative of arterial pressure of the patient. The hemodynamic sensor 108 of FIG. 1 may include the hemodynamic sensor 300 and/or include one or more features thereof. The hemodynamic sensor 300 is one example of a minimally invasive hemodynamic sensor that can be attached to the patient 104 via, e.g., a radial arterial catheter inserted into an arm of the patient. In other examples, the hemodynamic sensor 300 can be attached to the patient 104 via a femoral arterial catheter inserted into a leg of the patient.

As illustrated, the hemodynamic sensor 300 includes a housing 318, a fluid input port 320, a catheter-side fluid port 322, and an I/O cable 324. The fluid input port 320 is configured to be connected via tubing or other fluidic (e.g., hydraulic) connection to a fluid source, such as a saline bag or other fluid input source. The catheter-side fluid port 322 is configured to be connected via tubing or other fluidic connection to a catheter (e.g., a radial arterial catheter or a femoral arterial catheter) that is inserted into an arm of the patient (i.e., a radial arterial catheter) or a leg of the patient (i.e., a femoral arterial catheter). The I/O cable 324 may be configured to connect to a hemodynamic monitor (e.g., the graphical user interface 132) via, e.g., one or more of I/O connectors in the monitor. The housing 318 of the hemodynamic sensor 300 can contain (e.g., enclose) one or more pressure transducers, communication circuitry, processing circuitry, and/or corresponding electronic components to sense fluid pressure corresponding to an arterial pressure of the patient. One or more of these may be transmitted to the hemodynamic monitor (e.g., the graphical user interface 132) via the I/O cable 324.

In operation, a column of fluid (e.g., saline solution) can be introduced from a fluid source (e.g., a saline bag) through the hemodynamic sensor 300 via fluid input port 320 to catheter-side fluid port 322 toward the catheter inserted into the patient. The arterial pressure is communicated through the fluid column to pressure sensors located within housing 316 which sense the pressure of the fluid column. The hemodynamic sensor 300 translates the sensed pressure of the fluid column to an electrical (e.g., analog) signal via the pressure transducers and outputs the corresponding electrical signal. The hemodynamic sensor 300 can therefore transmit, to the endotype analysis system 114 (e.g., via the signal converter 112), analog sensor data (or a digital representation of the analog sensor data) that is representative of real-time, perhaps even beat-to-beat, monitoring of the arterial pressure of the patient.

FIG. 4 is a perspective view of an example hemodynamic sensor 426 for sensing hemodynamic data representative of arterial pressure of the patient. The hemodynamic sensor 426 is an example of a non-invasive hemodynamic sensor that can be attached to the patient via one or more finger cuffs to sense data representative of arterial pressure of the patient. The hemodynamic sensor 426 includes an inflatable finger cuff 428 and a heart reference sensor 430. The inflatable finger cuff 428 can include an inflatable blood pressure bladder configured to inflate and deflate as controlled by a pressure controller (not illustrated) that may be pneumatically connected to the inflatable finger cuff 428. The inflatable finger cuff 428 can additionally or alternatively include an optical (e.g., infrared) transmitter and/or an optical receiver that are electrically connected to the pressure controller (not illustrated). The optical transmitter and the optical receiver can measure the changing volume of the arteries under the cuff in the finger. The optical transmitter and the optical receiver can be positioned to transmit and receive light therebetween through the inflatable blood pressure bladder.

In operation, the pressure controller may continually adjust pressure within the finger cuff to maintain a constant volume of the arteries in the finger (e.g., an unloaded volume of the arteries) as measured via the optical transmitter and optical receiver of inflatable finger cuff 428. The pressure applied by the pressure controller to continuously maintain the unloaded volume can be representative of the blood pressure in the finger and can be communicated by the pressure controller to the endotype analysis system (e.g., the endotype analysis system 114). The heart reference sensor 430 can measure the hydrostatic height difference between the level at which the finger is kept and a reference level for the pressure measurement, which typically is a heart level. Accordingly, the hemodynamic sensor 426 transmits sensor data that is representative of substantially continuous beat-to-beat monitoring of the arterial pressure waveform of the patient. As noted above, this sensor data can be used to extract an arterial pressure signal waveform 200 (e.g., the arterial pressure signal waveform 200) and/or one or more heart health parameters therefrom.

### Endotype Determination

The endotype analysis system 114 can be configured to identify one or more of the heart health parameters relevant to identification of an endotype of hypotension and/or to determine the endotype of hypotension itself. The heart health parameters can include health parameters that can be measured from the arterial pressure signal waveform (e.g., the arterial pressure signal waveform 200) and/or that may be useful in identifying (e.g., diagnosing) an endotype of hypotension. There are many potential heart health parameters that may be extracted from the arterial pressure signal waveform, but they can include, for example, cardiac output (CO), stroke volume (SV), stroke volume variation (SVV), diastolic pressure (DIA), pulse rate (PR), stroke volume index (SVI), systemic vascular resistance (SVR), mean arterial pressure (MAP), HPI, and/or others. Additionally or alternatively, the heart health parameters can include systemic vascular resistance index (SVRI), cardiac index (CI), and/or systolic pressure (SYS).

The endotype analysis system 114 may receive the converted data (e.g., the heart health parameters in digital form) from the signal converter 112 via the hemodynamic data interface 118. The endotype analysis system 114 may then transmit the heart health parameters to the machine learning model 124 and/or the unsupervised training module 128.

The machine learning model 124 can be configured to receive the heart health parameters and encode them into different health parameters. For example, the encoded health parameters may be health parameters in a different space, such as a latent space. As an example, the endotype analysis system 114 can encode a plurality of heart health parameters into one, two, three, four, or more latent space heart health parameters. In some embodiments, the endotype analysis system 114 can convert the heart health parameters into a number of latent space heart health parameters that is less than or at most half of the number of heart health parameters in the original space. The endotype analysis system 114 can determine or generate a location of those one or more latent space heart health parameters. This location may be in latent space. The location may be based on the encoding of the original heart health parameters. For two latent space heart health parameters, a two-dimensional location can correspond to a coordinate location within a plot of the two latent space heart health parameters.

The machine learning model 124 can determine a relative location of the latent space heart health parameters. The relative location of the latent space heart health parameters can correspond to and/or represent a particular cluster of latent space heart health parameters that corresponds to the particular determined latent space heart health parameters. In some embodiments, the machine learning model 124 can additionally or alternatively determine a relative location of the original (e.g., decoded or non-encoded) heart health parameters in the original space.

To determine a relative location of latent space heart health parameters corresponding to the received arterial pressure signal waveform in latent space, the machine learning model 124 can transmit the latent space heart health parameters to a learning algorithm, such as the unsupervised training module 128. The unsupervised training module 128 can determine locations (e.g., in latent space) of other arterial pressure signal waveforms, such as from a plurality of patients who can serve as reference patients for the endotype analysis system 114.

The endotype analysis system 114 may obtain a plurality of reference heart health parameters from corresponding reference arterial pressure signal waveforms. For example, the endotype analysis system 114 may receive a plurality of reference analog hemodynamic sensor signals from a plurality of reference patients. One or more signal converters (e.g., the signal converter 112) can convert the plurality of reference analog hemodynamic sensor signals to a corresponding plurality of reference arterial pressure signal waveforms. The endotype analysis system 114 can extract corresponding sets of reference heart health parameters from the plurality of reference arterial pressure signal waveforms. The sets of reference heart health parameters can each include one or more heart health parameters described herein, such as, for example, a stroke volume index (SVI), a heart rate (HR), a cardiac index (CI), a systemic vascular resistance index (SVRI), and/or a stroke volume variation (SVV). The endotype analysis system 114 may use the machine learning model 124 to encode the plurality of reference sets of heart health parameters into a different plurality (e.g., first and second) reference latent space heart health parameters.

### Autoencoding Data in Latent Space

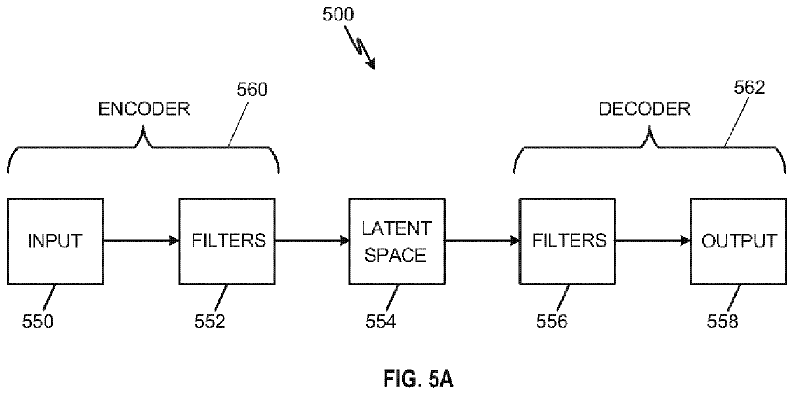

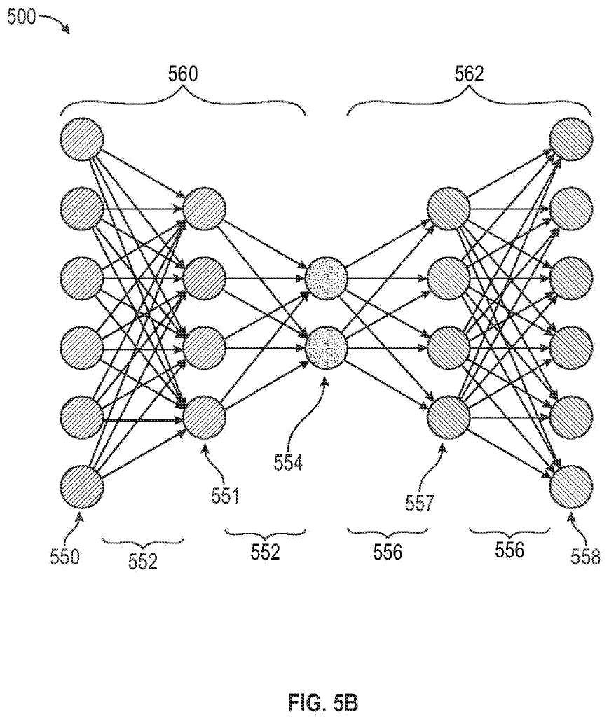

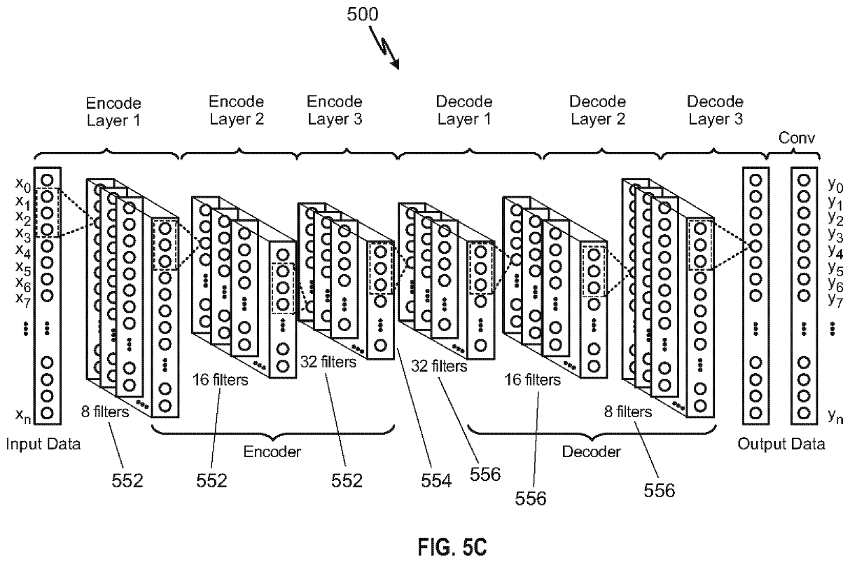

As noted above, the endotype analysis system 114 can include a machine learning model (e.g., a trained machine learning model), such as an autoencoder. FIG. 5A is a block diagram illustrating an example autoencoder model 500 of an endotype analysis system (e.g., the endotype analysis system 114), according to some embodiments. The autoencoder model 500 can include input data 550, one or more encoding filters 552, a latent space 554, one or more decoding filters 556, and output data 558. The encoder 560 can include the input data 550 and/or the encoding filters 552. Additionally or alternatively, the decoder 562 can include the decoding filters 552 and/or the output data 558.

The autoencoder model 500 can be a machine learning model. For example, the autoencoder model 500 can be a deep-learning-based model that is trained or learning to use neural network architecture to convert the heart health parameters into latent space heart health parameters. The input data 550 of the autoencoder model 500 can include one or more heart health parameters (e.g., in digital form) and/or one or more arterial pressure signal waveforms. The input data 550 may be received at regular and/or irregular intervals. The input data 550 may be received about every 5 s, about every 10 s, about every 20 s, about every 30 s, about every 45 s, about every 1 min, about every 2 min, about every 5 min, about every 10 min, about every 15 min, about every 20 min, about every 30 min, about every 1 hour, any value therein, or fall within a range having endpoints therein. For example, in some embodiments, the input data 550 is received about every 15 minutes.

The input data 550 can be encoded via one or more encoding filters 552 of the autoencoder model 500 into a condensed version of data in a different data space, such as a latent space 554. The latent space 554 can store data in a condensed form or format. The condensed data in latent space 554 can be then decoded via one or more decoding filters 552 of the autoencoder model 500 into output data 558. The output data 558 can be the original one or more heart health parameters and/or the one or more arterial pressure signal waveforms. The autoencoder model 500 can compare the output data 558 with the input data 550 to confirm that no relevant data was lost during the filtering by any of the encoding filters 552 and/or of the decoding filters 556. The autoencoder model 500 can be tuned to remove extraneous or superfluous data that is not necessary for determining an endotype of hypotension. The encoder 560 may refer to a portion of autoencoder model 500 that encodes, or compresses, the input data 550 into a smaller number of variables that include all of the most relevant information of the input data 550, using the one or more encoding filters 552. The decoder 562 can take the compressed encoded information and expand it to create the output data 558, using the one or more decoding filters 556.

FIG. 5B shows an example autoencoder model 500 using fully connected encoding and decoding layers. FIG. 5C shows an example autoencoder model 500 using convoluted encoding and decoding layers. Reference will be made to both figures, except where specifically noted. The encoding filters 552 and decoding filters 556 of the encoder 560 and the decoder 562, respectively. The encoding filters 552 of encoder 560 can include a plurality of encoding filters 552. Additionally or alternatively, the decoding filters 552 can include a plurality of decoding filters 552.

The encoding filters 552 can perform mathematical operations that transform the input data 550 into compressed data. Data samples of each element (e.g., heart health parameter) of the input data 550 can be reduced by a target encoding factor by each of the encoding filters 552 until fully compressed data reaches the latent space 554. The decoding filters 556 can be applied to the compressed data in latent space 554 to yield output data 558. Data samples may be expanded by a target decoding factor (e.g., different from the target encoding factor) by each of the decoding filters 556 until resulting in the output data 558. An algorithm within autoencoder model 500 can autogenerate the architecture of encoding filters 552 and/or the decoding filters 556.

As shown in FIG. 5B, the architecture may include fully connected layers where each of the one or more encoding filters 552 and/or each of the one or more decoding filters 556 receives the same number of data points as was output from the previous layer. In some embodiments, the autoencoder model 500 includes only fully connected layers and/or does not include any convolutional layers. The fully connected deep learning autoencoder model 500 of FIG. 5B may be referred to as a dense or feedforward neural network. As shown, each node in one layer may be connected to every node in the subsequent layer. Additionally or alternatively, the output from each node can serve as an input to every node in the subsequent layer. Each node can represent a corresponding input value, such as a heart health parameter or a feature thereof. The autoencoder model 500 can include one or more hidden layers between the input data 550 and the latent space 554. Additionally or alternatively, the autoencoder model 500 can include one or more hidden layers between the latent space 554 and the output data 558. In one or more of these hidden layers, each node can be fully connected with nodes of a precedent and/or a subsequent layer. Each layer can apply a weight or weighting to data for each node. This weight may be learned during a training phase of the model. Each hidden layer may apply one or more activation functions to the received interim data (e.g., interim encoded data 551, interim decoded data 557) from the precedent layer. One or more of the activation functions may be a non-linear function, such as a trigonometric function. In some embodiments, the activation function includes a hyperbolic tangent (tanh) function.

During training of the autoencoder model 500, weights of each connection between layers (and/or of the activation functions and/or values thereof themselves) may be adjusted by backpropagating and/or gradient descent. The autoencoder model 500 can adjust these connection weights based on a goal of reducing or even minimizing a difference between a predicted output and an actual output.

As shown in FIG. 5C, the autoencoder model 500 can include a convolutional neural network with auto-selected convolutions and pooling, based on the input data 550 and target output data 558. Complex nonlinear relationships may exist among filtered data at each of the one or more layers of encoding filters 552 and/or decoding filters 556. In the example of FIG. 5C, the filters of the encoding filters 552 and the decoding filters 556 have an 8-56-32 architecture. A first layer of encoding filters 552 has 8 filters, a second layer of encoding filters 552 has 56 filters, and a third layer of encoding filters 552 has 32 filters. As shown, a first layer of decoding filters 552 has 32 filters, a second layer of decoding filters 552 has 56 filters, and a third layer of decoding filters 552 has 8 filters. In the example of FIG. 5C, the autoencoder model 500 has 12,000 parameters and requires 112 convolution operations. Parameters may be determined as the autoencoder model 500 is or becomes trained. Once trained, the encoding filters 552 can enable the compression and encoding of the input data 550 (e.g., heart health parameters, arterial pressure signal waveforms). Additionally or alternatively, once trained the decoding filters 552 can enable the expansion and decoding of the compressed data (e.g., latent space heart health parameters) into the output data 558 (e.g., the heart health parameters, the arterial pressure signal waveforms).

The autoencoder model 500 can be used in connection with an unsupervised training module (e.g., the unsupervised training module 128) to conduct further analysis on the latent space data (e.g., the latent space heart health parameters) in the latent space 554 before the latent space data is decoded into the output data 558.

### Latent Space Location Determination

The unsupervised training module 128 can receive this different plurality of reference latent space heart health parameters to generate reference locations in latent space for each of the plurality of reference arterial pressure signal waveforms. The unsupervised training module 128 can use one or more criteria (e.g., clustering evaluation criteria or cluster evaluation metrics) or sets of such criteria to determine how these latent space heart health parameters should be located. Using clustering evaluation criteria or cluster evaluation metrics may include determining a goodness of clustering among a plurality of data points in latent space. In some embodiments, the unsupervised training module 128 can use one or more of the same clustering evaluation criteria or cluster evaluation metrics to determine a goodness of clustering in the original space among a plurality of data points corresponding to the latent space data points. This may be done, for example, to validate the goodness of clustering in the latent space.

For example, in some embodiments the unsupervised training module 128 can use silhouette criteria (e.g., a silhouette score) to determine the reference locations of the latent space heart health parameters. The unsupervised training module 128 can determine the silhouette criteria to identify a goodness of clustering of the latent space heart health parameters and/or their reference locations. Silhouette criteria can include metrics used to assess the validity and quality (e.g., goodness) of clusters in a clustering algorithm. Each reference location can obtain a silhouette score based on the silhouette criteria. The silhouette score can indicate how similar a reference location is to its own cluster compared to other clusters. The silhouette score may range from −1 to 1, where a high value indicates that the reference location is well matched to its own cluster and poorly matched to nearby or neighboring clusters.

A silhouette score s(i) can be given as

$$s ( i ) = \frac{( b ( i ) - a ( i ) )}{max \{ a ( i ) , b ( i ) \}}$$

where a(i) represents the average distance from the ith data point to the other data points in the same cluster, and b(i) represents the smallest average distance from the ith data point to data points in a different cluster, minimized over clusters. An overall silhouette score for the clustering can be the average of the silhouette score for each instance.

A score close to 1 indicates that the reference location is well matched to its own cluster and poorly matched to neighboring clusters. A score around 0 indicates overlapping clusters. A score close to −1 indicates that the reference location may be assigned to the wrong cluster. The unsupervised training module 128 can use the silhouette criteria to evaluate how well-defined and distinct the clusters corresponding to the latent space heart health parameters are. In some embodiments, the endotype analysis system 114 can automatically test different numbers of clusters and use silhouette scores to identify a best or optimal number (e.g., max number) of clusters associated with the reference locations. FIG. 6A shows a graph of a results of calculating a goodness of clustering across a potential number of clusters in latent space, ranging from 2 to 14 clusters. As shown, for the input data (e.g., input data 550) used, a max score or max number of clusters was determined to be 4 clusters. This max score indicates that the best number of clusters is likely 4 clusters.

The unsupervised training module 128 can associate each of the reference arterial pressure signal waveforms to a corresponding cluster in latent space. This may be done based on the best number of clusters previously identified. In this way, the unsupervised training module 128 can determine a set of clusters or reference clusters that can be used to test future latent space heart health parameters.

The unsupervised training module 128 can determine a cluster associated with each of the reference arterial pressure signal waveform in latent space based on the set of clusters and/or on the reference locations in latent space of each of the plurality of reference arterial pressure signal waveforms. The endotype analysis system 114 can then use the cluster information associated with the reference arterial pressure signal waveforms to determine a corresponding endotype of hypotension of each of the reference patients. In some embodiments, the unsupervised training module 128 does not determine any clustering evaluation criteria other than the silhouette criteria.

In some embodiments, the unsupervised training module 128 can additionally or alternatively calculate a Calinski-Harabasz index to assess the goodness of the clusters in the clustering algorithm. The Calinski-Harabasz (CH) index, or Variance Ratio Criterion, describes the ratio of the between-cluster variance to the within-cluster variance.

A CH index can be given by

$$CH = \frac{Between cluster variance}{Within cluster variance} \times \frac{Number of clusters - 1}{Number of data points - Number of clusters}$$

A higher CH index can indicate better-defined, more separate clusters. The endotype analysis system 114 can calculate the CH index for different numbers of clusters (k values) of the reference locations and select the number of clusters that maximizes the index. The peak in the CH index may suggest an optimal number of clusters. In some embodiments, the endotype analysis system 114 can compare the results of the silhouette criteria and the CH index to determine an optimal or best number of clusters.

In some embodiments, the unsupervised training module 128 can additionally or alternatively calculate a Davies-Bouldin Index (DBI) to generate a clustering validation metric. The DBI can also be used to evaluate the quality of clusters among the reference locations. The DBI can include an indication of the compactness and separation among clusters. Generally, the lower the Davies-Bouldin Index, the better the clustering.

A DBI can be calculated by finding a cluster centroid of each cluster of reference locations. Each cluster has its own centroid. A centroid can refer to the mean of all the data points in that cluster. The unsupervised training module 128 can calculate a similarity between each cluster and every other cluster. This similarity may be based on a distance metric (e.g., Euclidean distance) and can represent how close or similar the clusters are.

For each cluster, a Rᵢ may be calculated, which is given by

$${R}_{i} = \frac{{compactness}_{i} + {compactness}_{j}}{{separation}_{ij}}$$

where compactness; represents the compactness of cluster i, compactness, represents the compactness of cluster j, and separationᵢⱼ represents the separation between cluster i and cluster j. Thus, a DBI can be given by

$$DBI = \frac{1}{k} {\sum }_{i = 1}^{k} {R}_{i}$$

The unsupervised training module 128 can use any combination of the criteria described above and/or others to determine a goodness of clustering of the reference locations and, from the goodness, a best number of clusters of reference locations. In some embodiments, only silhouette criteria are used. In other embodiments, two or more sets of criteria and/or indices are used.

In some embodiments, the unsupervised training module 128 can additionally or alternatively use a Gaussian Mixture Model (GMM). A GMM is a probabilistic model that can represent a mixture of multiple Gaussian (e.g., normal) distributions. As with the other criteria and indices described above, the GMM can be used for estimating an amount or goodness of clustering and-or density information associated with each cluster. The unsupervised training module 128 can calculate an overall distribution of the data as a combination of several Gaussian distributions, each associated with a different cluster of reference locations.

The unsupervised training module 128 can identify a number of clusters for the model and calculate means, covariances, and/or weights for each cluster. These can correspond to the center, shape, and relative importance of each Gaussian distribution. The unsupervised training module 128 can then generate an Expectation-Maximization (EM). To do this, the unsupervised training module 128 can determine a probability of each reference location belonging to a particular cluster by calculating a Bayesian posterior probability. The unsupervised training module 128 can maximize the parameters (e.g., means, covariances, and weights) of each cluster to maximize a likelihood of the reference locations in latent space. The unsupervised training module 128 can take a weighted average of the reference locations based on the posterior probabilities obtained. This process may be iterated until a convergence is reached. Convergence may be determined when a change in parameters between iterations falls below a convergence threshold and/or after a maximum number of iterations has been reached. Based on the extent of the convergence, the unsupervised training module 128 can assign each location (e.g., reference location) in latent space to a particular cluster based on a highest posterior probability.

In some embodiments, the endotype analysis system 114 (e.g., the machine learning model 124) can further decode the latent space heart health parameters back into corresponding heart health parameters. The machine learning model 124 can include a fully connected deep learning model, a convoluted deep learning model, and/or some other type of learning model. In some embodiments, one or more of these heart health parameters can be transmitted to the graphical user interface 132 for display to a healthcare professional.

In some embodiments, the endotype analysis system 114 can validate a goodness of clustering in the latent space by determining a goodness of clustering in the original space and comparing the two scores. If the goodness of clustering in the original space and in the latent space indicate the same optimal or best number of clusters, then the endotype analysis system 114 can validate the best number of clusters in the latent space. The endotype analysis system 114 may validate the best number of clusters in the latent space by determining the best number of clusters in the original space by applying one or more of the clustering evaluation metrics described herein. Accordingly, decoding the latent space heart health parameters to the heart health parameters in the original space can be done to validate the encoding of the machine learning model 124. FIG. 6B shows a graph of an example evaluation of a goodness of clustering of a set of locations in original space using a clustering evaluation metric. As shown, the evaluation resulted in a best number of clusters as four clusters. When comparing FIG. 6B with FIG. 6A, one can see that both the original space and the latent space goodness of clustering arrived at the same best number of clusters. Accordingly, FIG. 6B can serve as a validation of the best number of clusters found in FIG. 6A.

FIG. 6C shows a graph of example results from an arterial pressure signal waveform cluster determination. As shown, a plurality of locations have been plotted within a Cartesian coordinate of first and second latent space heart health parameters. Such a plot could be made in 3, 4, 5, 6, or more dimensions, but for clarity of illustration two latent space heart health parameters have been used in FIG. 6C. As shown, each location has been associated with a corresponding cluster among four clusters, based on the cluster determination described above. The four clusters are a result of the evaluation of the goodness of clustering shown in FIGS. 6A-6B. Once the best or optimal number of clusters has been identified, the endotype analysis system 114 can determine which locations are associated with which cluster (e.g., what the relative locations of each location are within the latent space). As shown, a number of reference patients associated with a first cluster is 2434, a number of reference patients associated with a second cluster is 1851, a number of reference patients associated with a third cluster is 1635, and a number of reference patients associated with a fourth cluster is 928. Each cluster is associated with a corresponding endotype of hypotension.

FIG. 6D shows a plurality of box-and-whisker plots of example results of a trained machine learning model that is configured to diagnose or determine an endotype of hypotension of a test patient. As shown, four endotypes of hypertension have been identified. These four endotypes were determined using the cluster optimization described above with respect to FIGS. 6A-6C. FIG. 6D shows the names of the four identified endotypes of hypotension that were identified: Vasodilation, Myocardial Depression, Bradycardia, and Hypovolemia. For each identified endotype, a box-and-whisker plot indicates a normalized distribution of five heart health parameters (in original space) among the reference patients who were associated with the corresponding endotype. These five heart health parameters (stroke volume index (SVI), heart rate (HR), cardiac index (CI), systemic vascular resistance index (SVRI), and stroke volume variation (SVV)) were the five heart health parameters used to calculate the endotypes in the example analysis conducted and shown in FIGS. 6A-6D. In some embodiments, the system does not use any other heart health parameters other than these five heart health parameters.

As shown, the 25th-75th percentile distributions of each of the five heart health parameters indicates that Vasodilation patients tend to have a below-average systemic vascular resistance index (SVRI) and stroke volume variation (SVV). These factors suggest that for Vasodilation patients, vasoconstriction of their arteries and veins and/or additional hydration may be beneficial in improving any diagnosis or potential diagnosis of hypotension.

As shown, the 25th-75th percentile distributions of each of the five heart health parameters indicates that Myocardial Depression patients tend to have a below-average stroke volume index (SVI) and cardiac index (CI), but a normal stroke volume variation (SVV). These factors suggest that for Myocardial Depression patients, a patient may need to receive vasopressors to constrict blood vessels and increase blood pressure and/or to receive inotropic medications to improve the contractions of the heart.

As shown, the 25th-75th percentile distributions of each of the five heart health parameters indicates that Bradycardia patients tend to have a below-average heart rate (HR), cardiac index (CI), and stroke volume variation (SVV). These factors suggest that for Bradycardia patients, the patient may need to receive atropine to block the effects of the vagus nerve and/or to receive temporary or permanent pacing of the heart's electrical conduction system. In some cases, dopamine and/or norepinephrine may be used.

As shown, the 25th-75th percentile distributions of each of the five heart health parameters indicates that Hypovolemia patients tend to have a below-average stroke volume index (SVI) and above-average heart rate (HR) and stroke volume variation (SVV). These factors suggest that for Hypovolemia patients, the patient may need to receive delivery of intravenous fluid (e.g., saline), possibly a blood transfusion, and/or perhaps receive vasopressors to raise blood pressure.

As described herein, one or more of these results of the analysis shown in FIGS. 6A-6D may be transmitted to a user interface (e.g., the graphical user interface 132) as an alert. For example, in some embodiments, the determined endotype (e.g., vasodilation, myocardial depression, bradycardia, hypovolemia, etc.) may be displayed as an alert. Additionally or alternatively, the alert may include one or more of the determined heart health parameters (e.g., SVI, HR, CI, SVRI, SVV). For example, in some embodiments, the display may also include a plot of one or more of the heart health parameters within a plot like the box-and-whiskers plot of FIG. 6D. Whether and/or which heart health parameters are displayed may depend on the heart health parameters that require the most urgent attention and/or which deviate most from a normalized value, as compared to healthy patients, compared to hypotensive patients, and/or compared to other patients having the determined endotype of hypotension. Accordingly, the displayed heart health parameters may depend on whether a corresponding heart health parameter satisfies or exceeds a threshold.

Additional user interfaces are described below, with respect to FIGS. 6E-6K.

The threshold may include an urgency threshold (e.g., a time threshold). Additionally or alternatively, the threshold may include an extremity threshold (e.g., a normalized value threshold, a normalized value deviation threshold). An example extremity threshold may include by how much a given heart health parameter deviates from the 25th 75th percentile of a determined set of reference patients (such as the one shown in FIG. 6D). In some embodiments, alert may include an audible indication (e.g., one or more tones, pulsing tones, tones increasing in volume), a visual indication (e.g., blinking words, pictures, or other indicators; enlarged or highlighted words or pictures), and/or a haptic indication (e.g., a vibration of a certain frequency, a change in vibration frequency or duration, a pattern of vibration, etc.). Other types of alarms described above are also possible.

FIGS. 6E-6K illustrate example user interfaces which may be used by a medical professional to analyze medical information associated with a patient. In some embodiments, the user interfaces may represent the graphical user interface 132 described above. The user interfaces may be presented via a display of a system, such as a computer, laptop, tablet, mobile device, and so on. As will be described, the user interfaces may be responsive to user input such that a medical professional can interact with the user interfaces. For example, a medical professional may provide input to view detailed information associated with a particular patient. As another example, a medical professional may provide input to view different visualizations of endotypes for patients or a particular patient. For this example, the medical professional may cycle between the user interfaces illustrated in FIGS. 6E-6K.

FIGS. 6E-6K specifically describe techniques to visualize an endotype trend over time which is associated with a patient (e.g., in the context of endotype clusters obtained previously from a database of a plurality of patients). As described above, measurements or input data associated with a patient may be obtained at different times. Example measurements or input data described herein may include hemodynamic signal measurements, heart health parameters, arterial pressure signal waveforms, and so on. These measurements or input data may be obtained at periodic or non-periodic intervals (e.g., every 5 seconds, every 2 hours, a few times a day, a few times a week, and so on). Using these measurements or input data, for example as described in FIG. 1, an endotype of hypotension for the current patient may be determined. In this way, the current patient's hypotension may be monitored over time. As may be appreciated, hypotensive events may adjust in character over time such that a patient's determined endotype may correspondingly adjust. FIGS. 6E-6K illustrate different techniques by which such endotype adjustment over time (referred to herein as an endotype trend) may be succinctly presented in substantially real-time to the medical professional.

In some embodiments a medical professional may select a patient to review in detail, using the data stored for a particular patient. For example, the medical professional may provide input indicative of the patient (e.g., the patient's name, ID number, and so on). The system described herein may access latent space heart health parameters for the patient which were determined at different points in time. For example, the latent space heart health parameters may be selected for a previous threshold amount of time. In this example, the medical professional may optionally indicate the threshold amount of time such that the medical professional can view the patient's determined endotypes during the threshold amount of time (e.g., a previous hour, a previous day, a previous week, and so on).

The latent space heart health parameters may be presented in the user interface as reflecting an endotype trend associated with the patient in the context of endotype clusters and their associated probabilities as determined previously (e.g., from a database of a plurality of patients). For example, the endotype trend may graphically illustrate the determined endotypes of the patient either in real time or in off-line review over the previous threshold amount of time. In this way, and as an example, the user interface may reflect that the patient started as having vasodilation and ended with having hypovolemia. As another example, the user interface may reflect that the patient started as having vasodilation with lower likelihoods (e.g., probabilities) of bradycardia, hypovolemia, and myocardial depression. For this example, the user interface may additionally reflect that over time the patient still has vasodilation but with a higher likelihood of bradycardia, hypovolemia, or myocardial depression.

The endotype clusters may, in some embodiments, be determined using the techniques described herein. Thus, the clusters may be formed using information associated with a multitude of patients. Subsequently, information from a new patient may be accessed. For example, a medical professional may obtain measurements associated with the patient. These measurements may subsequently be mapped to latent space heart health parameters. Thus, in some embodiments the latent space heart health parameters may represent new information. The user interfaces described below may present (1) the previously determined clusters and (2) the new information for the patient. For example, the newly determined latent space heart health parameters may be mapped into the previously determined clusters (e.g., data points may be added to the clusters, with each data point representing a combination of latent space heart health parameters determined for the patient at a particular time).

While FIGS. 6E-6K illustrate example graphical representations of a patient's endotype trend over time, in some embodiments the user interface may include textual information. For example, the user interface may include a textual summary describing the endotype trend of the patient and the previously determined clusters from the database. As an example, the textual summary may describe, and optionally include quantitative information regarding, the patient having less vasodilation and more myocardial depression over time.

FIG. 6E illustrates an example user interface 650 of an endotype trend 652 associated with a patient. In the illustrated example, two latent dimensions are included as respective axes. Individual data points and/or time points (e.g., data points 654A-654B) for an individual patient are plotted in the user interface 650. These data points (e.g., time points) may represent latent space heart health parameters corresponding to a patient taken at a particular time or in real time, with the user interface 650 including a multitude of such data points for a multitude of patients from a database. The patients may share one or more similarities or characteristics with the current patient. The data points from the database patients are clustered into different endotypes (also referred to as data point clusters), such that a first portion of the data points are clustered into the vasodilation endotype (e.g., data point 654A), a second portion of the data points are clustered into the hypovolemia endotype (e.g., 654B), a third portion of the data points are clustered into the bradycardia endotype, and a fourth portion of the data points are clustered into the myocardial depression endotype.

With respect to the latent space heart health parameters (e.g., latent space dimensions), in the illustrated example each parameter may range from −1 to 1. In other embodiments the range of values may be different. As describe above, each data point may be mapped into this latent space based on input data associated with a patient. The data point may then be assigned an endotype, for example based on a likelihood of the assigned endotype exceeding likelihoods of the remaining endotypes determined based on the input data for the patients in the database. In some embodiments, this likelihood may be determined based on the input data and optionally may be determined using clustering techniques.

Thus, each of the data points depicted in user interface 650 may be assigned a respective endotype. In FIG. 6E, different colors are used to visually characterize a datapoint from the database as being assigned an endotype. For example, green may be used to represent vasodilation, red may be used to represent hypovolemia, purple may be used to represent bradycardia, and cyan may be used to represent myocardial depression. In some embodiments, a medical professional using user interface 650 may customize the colors which correspond to the endotypes. Optionally, the user interface may use other visual indicia, such as patterns, to represent the endotypes. In this way, the user interface 650 graphically illustrates the clustering of data points in the database as being associated with different endotypes.

The user interface 650 is presenting the above-described endotype trend 652 over time, which in this example is formed from four data points associated with the current patient (e.g., data points 652A-652D). Graphical elements (e.g., stars, circles, squares, points, etc.) may be presented for these data points 652A-652D. In some embodiments, different graphical elements may be used. To represent time passing, the graphical elements have different internally coloring. For example, the most recent data point may be colored opaquely (e.g., solid red). The data points which are further back in time may be colored more transparently. For example, the interior of the first of the stars (e.g., closer to the top of the figure) may be substantially transparent. Similarly, the recency may be used to adjust the color to a particular color (e.g., white) rather than to being transparent.

The user interface 650 thus renders the clustering actionable in that the medical professional can ascertain the patient's position within the user interface 650 over time within the context provided by the clustering of datapoints from the database. By way of example, the endotype trend 652 may indicate that the patient is trending from vasodilation to a different endotype.

In some embodiments, the user interface 650 may respond to user input selecting one of the data points 652A-652D which form the endotype trend 652. Upon selection, the user interface 650 may update to include detailed measurements or medical information associated with the patient for a particular time. In this way, the medical professional may succinctly access the detailed medical information for the patient.

FIG. 6F illustrates another example user interface 660 of the endotype trend 652 associated with the current patient. As illustrated, user interface 660 includes a multitude of shapes (e.g., ellipse 662). As will be described, each ellipse may include a subset of the above-described datapoints from the database which have substantially similar likelihoods of being assigned a particular endotype. While ellipses are shown in the example, as may be appreciated other shapes may be used. For example, a shape may follow an arbitrary contour line. In this example, the arbitrary contour line may be used to define which data points are associated with a similar likelihood of being assigned a particular endotype. Thus, the contour lines may define the extremities of arbitrary shapes (e.g., data blobs). User interface 660 may be accessed, in some embodiments, based on user input provided to user interface 650. For example, the above-described medical professional may toggle on the inclusion of the ellipses. User interface 660 may also be accessed directly, for example without viewing user interface 660.

Ellipse 662 is illustrated as encompassing a first portion of the data points (e.g., from the database) which are assigned as the hypovolemia endotype. The ellipse 662 may correspond to a probability area that indicates a likelihood of a corresponding endotype associated with the cluster. These data points may be associated with a similar likelihood of assignment. For example, the data points may be within a threshold likelihood percentage of each other. As another example, the ellipse may represent a range of likelihoods and the data points may extend between that range.

Ellipse 664 is illustrated as encompassing a second portion of the data points from the database. For example, the second portion may extend between 664 and 666. In the illustrated example, some of the data points in the second portion are assigned as the vasodilation endotype. These data points may have similar likelihood of being assigned the hypovolemia endotype. However, the data points may also have a higher likelihood of being assigned the vasodilation endotype. That is, the system described herein (e.g., the endotype analysis system 114) may have determined likelihoods of the data points reflecting each of the endotypes. Thus, the lower portion of ellipse 664 in FIG. 6F may reflect the highest likelihood being hypovolemia while the upper portion may reflect the highest likelihood being vasodilation.

The ellipses may be used, as an example, by the medical professional to determine overlap between endotype clusters. With respect to the endotype trend 652, the medical professional can determine that the current (e.g., most recent) data point 652D has an overlap between vasodilation and bradycardia. In this way, the medical professional can determine the increasing likelihood of bradycardia.

FIG. 6G illustrates another example user interface 670 of the endotype trend 652 associated with the patient. In the illustrated example, the coloring associated with data points from the database has been removed (e.g., from user interface 650). Instead, a substantially solid color (e.g., grey) has been included. The removal of the color may help the medical professional understand the endotype trend 652 (e.g., movement of the patient's mapping into the latent space over time).

FIG. 6H illustrates another example user interface 680 of the endotype trend 652 associated with the patient. In the illustrated example, the coloring associated with the data points from the database has been removed (e.g., from user interface 660). Similar to the above, the removal may help the medical professional understand the endotype trend 652. The user interface 680 includes the ellipses from user interface 660 along with the associated endotype colors.

With respect to FIGS. 6E-6H, in some embodiments the endotype trend over time 692 may represent a line or vector. Optionally, portions of the line or vector may be assigned colors based on the endotypes determined for the data points which form the trend 692. For example, the portion of the line or vector which represents data points earlier in time may be green. The line or vector may start to become purple as the closer the line or vector gets to the current (e.g., most recent) data point.

FIG. 6I illustrates another example user interface 690 of an endotype trend over time 692 associated with a current patient. In the illustrated example, the endotype trend 692 is a trendline which extends between A and B. Data points A and B may represent an earliest and a most recent data point associated with the patient. The data points which form the trendline may be associated with times between those of A and B. The trendline may be formed by different techniques, such as via mapping different linear and/or non-linear functions onto the positions of the data points associated with the patient.

FIG. 6J illustrates another example user interface 692 of an endotype trend 652 associated with a current patient. In the illustrated example, the endotype trend 652 shows another example set of time points, similar to FIGS. 6E-6I. The time points can be associated with indicators indicative of the time points relative other of the plurality of indicators. One or more of the indicators may indicate a relative time at which the time points were obtained. For example, earlier time points may be more transparent, a different color, and/or include some other visual indicator, which may be different from later time points. Multiple degrees or levels of the differences among different time points may be available.

FIG. 6K illustrates another example user interface 694 of a plurality of trends of various attributes associated with the time points in FIG. 6J. For example, the user interface 694 can include time points associated with a patient's MAP, HPI, SVR, SVV, Harmonic associated with the locations of the latent space points, SV, HR, CO, dpdt, and/or Gaussian associated with the locations of the latent space points. For example, the Harmonic may refer to a harmonic probability associated with the plurality of locations in latent space of the plurality of arterial pressure signal waveforms from the patient. Additionally or alternatively, the Gaussian may refer to a Gaussian distribution associated with the plurality of locations in latent space of the plurality of arterial pressure signal waveforms.

### Example Methods of Endotype Determination

The example routines or methods herein illustrate various implementations of systems described herein. The blocks of the routines illustrate example implementations, and in various other implementations various blocks may be rearranged, and/or rendered optional. Further, various blocks may be omitted from and/or added to the example routines below, and blocks may be moved between the various example routines.

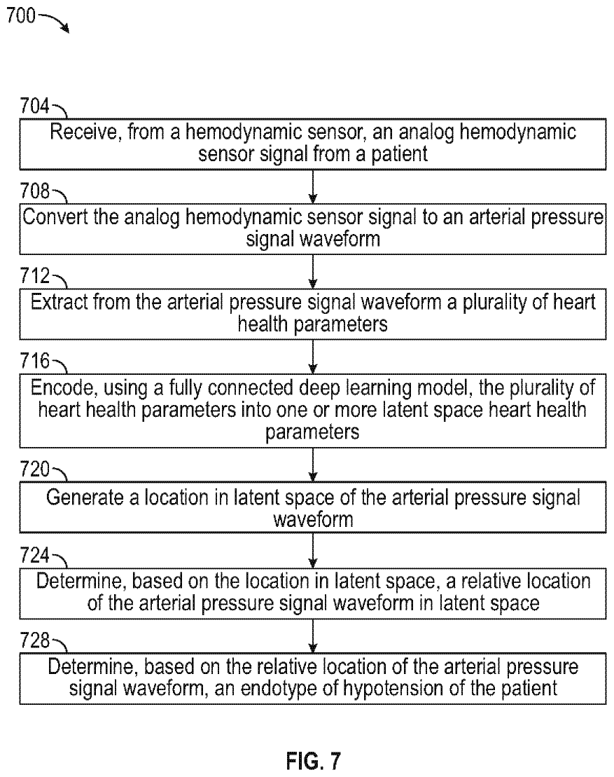

FIG. 7 shows a flowchart of an example method 700 for determining an endotype of hypotension of a patient. The method 700 can be performed by one or more systems described herein (e.g., the hemodynamic sensing system 100, the endotype analysis system 114, the machine learning model 124, the unsupervised training module 128, the autoencoder model 500, etc.) and/or a combination thereof. At block 704, the system can receive, from a hemodynamic sensor, an analog hemodynamic sensor signal from a patient. The analog hemodynamic sensor signal may correspond to a signal sensed by a hemodynamic sensor (e.g., hemodynamic sensor 108, the hemodynamic sensor 300, the hemodynamic sensor 426).

At block 708, the system can convert the analog hemodynamic sensor signal to an arterial pressure signal waveform. The arterial pressure signal waveform can correspond to one described herein, such as the arterial pressure signal waveform 200. At block 712, the system can extract, from the arterial pressure signal waveform, a plurality of heart health parameters. The heart health parameters can correspond to any heart health parameters described herein, such as, for example, cardiac output (CO), stroke volume (SV), stroke volume variation (SVV), diastolic pressure (DIA), pulse rate (PR), stroke volume index (SVI), systemic vascular resistance (SVR), mean arterial pressure (MAP), HPI, systemic vascular resistance index (SVRI), cardiac index (CI), systolic pressure (SYS), and/or any others described herein. In some embodiments, the system may extract a combination of SVI, HR, CI, SVRI, and SVV from the arterial pressure signal waveform, but other combinations are possible.

At block 716, the system can encode, using a fully connected deep learning model, the plurality of heart health parameters into one or more latent space heart health parameters. The fully connected deep learning model may include the autoencoder model 500, the machine learning model 124, and/or include one or more features of them. The latent space heart health parameters may be fewer in number than the number of heart health parameters extracted from the arterial pressure signal waveform. In some embodiments, the number of latent space heart health parameters is two.

At block 720, the system can generate a location in latent space of the arterial pressure signal waveform. The location can be based on the encoded latent space heart health parameters, such as on the values of the same. For example, if the number of latent space heart health parameters is two, the location may correspond to a coordinate location in a Cartesian plane. The system can, at block 724, determine a relative location of the arterial pressure signal waveform in latent space based on the location in latent space. The relative location may be indicative of a cluster associated with the arterial pressure signal waveform. The relative location and/or the associated cluster can be determined by an unsupervised training module (e.g., the unsupervised training module 128). The relative location and/or the associated cluster may be determined using one or more cluster evaluation metrics or criteria described herein (e.g., silhouette criteria, Calinski-Harabasz Index, and Davies-Bouldin Index, etc.). The clustering evaluation metric or criteria can be configured to indicate a goodness of clustering of the reference locations.

At block 728, the system can determine, based on the relative location and/or associated cluster of the arterial pressure signal waveform, an endotype of hypotension of the patient. In some embodiments, the system can generate, based on the determined endotype of hypotension of the patient, data for displaying an alert indicating the endotype of hypotension of the patient and/or one or more of the heart health parameters.

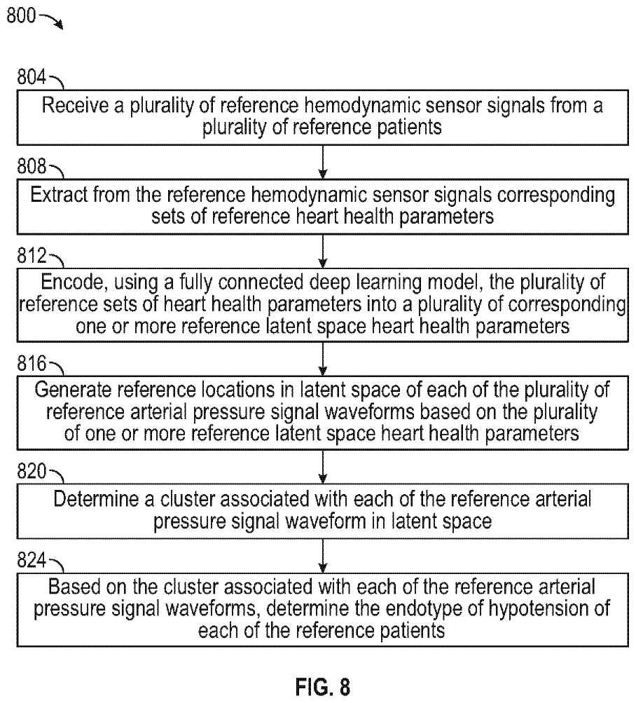

FIG. 8 shows a flowchart of an example method 800 for training a model to determine an endotype of hypotension of a patient. The method 800 can be performed by one or more systems described herein (e.g., the hemodynamic sensing system 100, the endotype analysis system 114, the machine learning model 124, the unsupervised training module 128, the autoencoder model 500, etc.) and/or a combination thereof.

At block 804 the system can receive a plurality of reference hemodynamic sensor signals from a plurality of reference patients. At block 808 the system can extract from the reference hemodynamic sensor signals corresponding sets of reference heart health parameters. The reference heart health parameters may include one or more heart health parameters described above with respect to FIG. 7. At block 812 the system can encode, using a fully connected deep learning model, the plurality of reference sets of heart health parameters into a plurality of corresponding one or more reference latent space heart health parameters. The reference latent space heart health parameters may be used by the system at block 816 to generate reference locations in latent space for each of the plurality of reference arterial pressure signal waveforms, based on the plurality of one or more reference latent space heart health parameters.

At block 820, the system can determine a cluster associated with each of the reference arterial pressure signal waveform in latent space. Each cluster may correspond to a relative location of the arterial pressure signal waveforms in latent space. Each cluster may be determined by determining a clustering evaluation metric, such as those described above, for each of the reference locations in latent space. Th system can determine a best number of clusters based on the clustering evaluation metric. In some embodiments, the system can associate, based on the best number of clusters, each of the reference arterial pressure signal waveforms to a corresponding cluster in latent space to determine a set of clusters.

At block 824, the system can determine the endotype of hypotension of each of the reference patients based on the cluster associated with each of the reference arterial pressure signal waveforms. Determining the endotype of hypotension may include determining, based on the set of clusters and/or on the reference locations in latent space of each of the plurality of reference arterial pressure signal waveforms, a cluster associated with each of the reference arterial pressure signal waveform in latent space. In some embodiments, the system can decode, using the fully connected deep learning model, the plurality of one or more reference latent space heart health parameters into the corresponding plurality of reference sets of heart health parameters. Based on the determined endotypes of hypotension, the system can generate the trained model for determining the endotype of hypotension of a patient, such as a test patient in an ICU, OR, and/or other patient care environment.

In some embodiments, the system comprises various features that are present as single features (as opposed to multiple features). For example, in one embodiment, the system includes a single hemodynamic sensor 108 with a single signal converter 112, a single processor 116 and a single memory 120, a single machine learning model 124 with a single unsupervised training module 128, as described herein. Multiple features or components are provided in alternate embodiments.

In some embodiments, the system (e.g., the hemodynamic sensing system 100) comprises one or more of the following: means for sensing heart pressure (e.g., the hemodynamic sensor 108, the hemodynamic sensor 300, the hemodynamic sensor 426), means for converting sensed data (e.g., the signal converter 112), and/or means for displaying data (e.g., the graphical user interface 132).

### Additional Example Implementations and Details Related to Computing Systems

In some implementations the systems described herein may comprise, or be implemented in, a “virtual computing environment”. As used herein, the term “virtual computing environment” should be construed broadly to include, for example, computer-readable program instructions executed by one or more processors to implement one or more aspects of the modules and/or functionality described herein. Further, in this implementation, one or more services/modules/engines and/or the like of the system may be understood as comprising one or more rules engines of the virtual computing environment that, in response to inputs received by the virtual computing environment, execute rules and/or other program instructions to modify operation of the virtual computing environment. For example, a request received from a user computing device may be understood as modifying operation of the virtual computing environment to cause the request access to a resource from the system. Such functionality may comprise a modification of the operation of the virtual computing environment in response to inputs and according to various rules. Other functionality implemented by the virtual computing environment (as described throughout this disclosure) may further comprise modifications of the operation of the virtual computing environment, for example, the operation of the virtual computing environment may change depending on the information gathered by the system. Initial operation of the virtual computing environment may be understood as an establishment of the virtual computing environment. In some implementations the virtual computing environment may comprise one or more virtual machines, containers, and/or other types of emulations of computing systems or environments. In some implementations the virtual computing environment may comprise a hosted computing environment that includes a collection of physical computing resources that may be remotely accessible and may be rapidly provisioned as needed (commonly referred to as “cloud” computing environment).

Implementing one or more aspects of the system as a virtual computing environment may advantageously enable executing different aspects or modules of the system on different computing devices or processors, which may increase the scalability of the system. Implementing one or more aspects of the system as a virtual computing environment may further advantageously enable sandboxing various aspects, data, or services/modules of the system from one another, which may increase security of the system by preventing, e.g., malicious intrusion into the system from spreading. Implementing one or more aspects of the system as a virtual computing environment may further advantageously enable parallel execution of various aspects or modules of the system, which may increase the scalability of the system. Implementing one or more aspects of the system as a virtual computing environment may further advantageously enable rapid provisioning (or de-provisioning) of computing resources to the system, which may increase scalability of the system by, e.g., expanding computing resources available to the system or duplicating operation of the system on multiple computing resources. For example, the system may be used by thousands, hundreds of thousands, or even millions of users simultaneously, and many megabytes, gigabytes, or terabytes (or more) of data may be transferred or processed by the system, and scalability of the system may enable such operation in an efficient and/or uninterrupted manner.

Various implementations of the present disclosure may be a system, a method, and/or a computer program product at any possible technical detail level of integration. The computer program product may include a computer-readable storage medium (or mediums) having computer-readable program instructions thereon for causing a processor to carry out aspects of the present disclosure.

For example, the functionality described herein may be performed as software instructions are executed by, and/or in response to software instructions being executed by, one or more hardware processors and/or any other suitable computing devices. The software instructions and/or other executable code may be read from a computer-readable storage medium (or mediums). Computer-readable storage mediums may also be referred to herein as computer-readable storage or computer-readable storage devices.

The computer-readable storage medium can be a tangible device that can retain and store data and/or instructions for use by an instruction execution device. The computer-readable storage medium may be, for example, but is not limited to, an electronic storage device (including any volatile and/or non-volatile electronic storage devices), a magnetic storage device, an optical storage device, an electromagnetic storage device, a semiconductor storage device, or any suitable combination of the foregoing. A non-exhaustive list of more specific examples of the computer-readable storage medium includes the following: a portable computer diskette, a hard disk, a solid state drive, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer-readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer-readable program instructions described herein can be downloaded to respective computing/processing devices from a computer-readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers, and/or edge servers. A network adapter card or network interface in each computing/processing device receives computer-readable program instructions from the network and forwards the computer-readable program instructions for storage in a computer-readable storage medium within the respective computing/processing device.

Computer-readable program instructions (as also referred to herein as, for example, “code,” “instructions,” “module,” “application,” “software application,” and/or the like) for carrying out operations of the present disclosure may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. Computer-readable program instructions may be callable from other instructions or from itself, and/or may be invoked in response to detected events or interrupts. Computer-readable program instructions configured for execution on computing devices may be provided on a computer-readable storage medium, and/or as a digital download (and may be originally stored in a compressed or installable format that requires installation, decompression, or decryption prior to execution) that may then be stored on a computer-readable storage medium. Such computer-readable program instructions may be stored, partially or fully, on a memory device (e.g., a computer-readable storage medium) of the executing computing device, for execution by the computing device. The computer-readable program instructions may execute entirely on a user's computer (e.g., the executing computing device), partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some implementations, electronic circuitry including, for example, programmable logic circuitry, field-programmable gate arrays (FPGA), or programmable logic arrays (PLA) may execute the computer-readable program instructions by utilizing state information of the computer-readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to implementations of the disclosure. It will be understood that each block of the flowchart illustrations and/or block diagrams, and combinations of blocks in the flowchart illustrations and/or block diagrams, can be implemented by computer-readable program instructions.

These computer-readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer-readable program instructions may also be stored in a computer-readable storage medium that can direct a computer, a programmable data processing apparatus, and/or other devices to function in a particular manner, such that the computer-readable storage medium having instructions stored therein comprises an article of manufacture including instructions which implement aspects of the function/act specified in the flowchart(s) and/or block diagram(s) block or blocks.

The computer-readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks. For example, the instructions may initially be carried on a magnetic disk or solid-state drive of a remote computer. The remote computer may load the instructions and/or modules into its dynamic memory and send the instructions over a telephone, cable, or optical line using a modem. A modem local to a server computing system may receive the data on the telephone/cable/optical line and use a converter device including the appropriate circuitry to place the data on a bus. The bus may carry the data to a memory, from which a processor may retrieve and execute the instructions. The instructions received by the memory may optionally be stored on a storage device (e.g., a solid-state drive) either before or after execution by the computer processor.

The flowcharts and block diagrams in the Figures illustrate the architecture, functionality, and operation of possible implementations of systems, methods, and computer program products according to various implementations of the present disclosure. In this regard, each block in the flowchart or block diagrams may represent a service, module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. In addition, certain blocks may be omitted or optional in some implementations. The methods and processes described herein are also not limited to any particular sequence, and the blocks or states relating thereto can be performed in other sequences that are appropriate.

It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions. For example, any of the processes, methods, algorithms, elements, blocks, applications, or other functionality (or portions of functionality) described in the preceding sections may be embodied in, and/or fully or partially automated via, electronic hardware such application-specific processors (e.g., application-specific integrated circuits (ASICs)), programmable processors (e.g., field programmable gate arrays (FPGAs)), application-specific circuitry, and/or the like (any of which may also combine custom hard-wired logic, logic circuits, ASICs, FPGAs, and/or the like with custom programming/execution of software instructions to accomplish the techniques).

Any of the above-mentioned processors, and/or devices incorporating any of the above-mentioned processors, may be referred to herein as, for example, “computers,” “computer devices,” “computing devices,” “hardware computing devices,” “hardware processors,” “processing units,” and/or the like. Computing devices of the above implementations may generally (but not necessarily) be controlled and/or coordinated by operating system software, such as Mac OS, IOS, Android, Chrome OS, Windows OS (e.g., Windows XP, Windows Vista, Windows 7, Windows 8, Windows 10, Windows 11, Windows Server, and/or the like), Windows CE, Unix, Linux, SunOS, Solaris, Blackberry OS, VxWorks, or other suitable operating systems. In other implementations, the computing devices may be controlled by a proprietary operating system. Conventional operating systems control and schedule computer processes for execution, perform memory management, provide file system, networking, I/O services, and provide a user interface functionality, such as a graphical user interface (“GUI”), among other things.

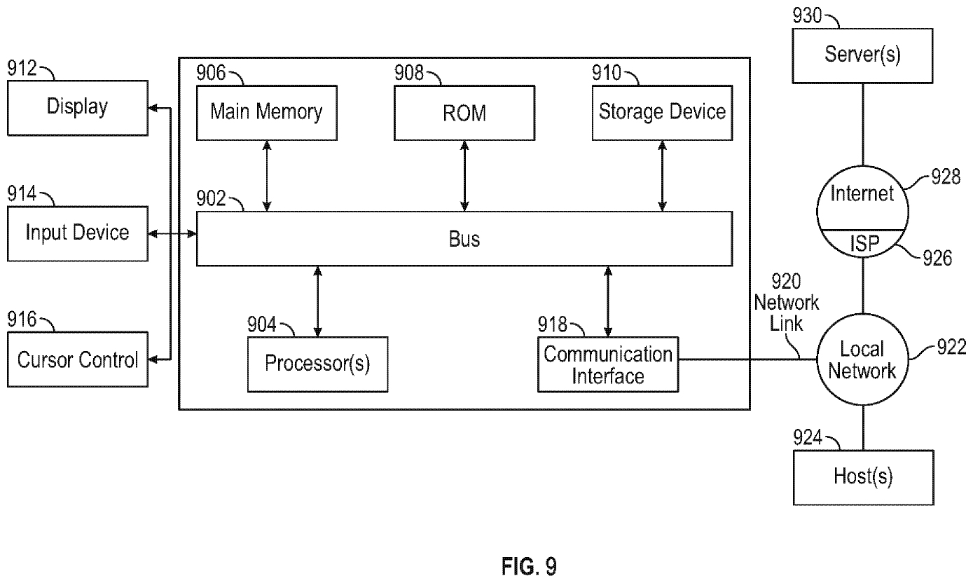

For example, FIG. 9 is a block diagram that illustrates a computer system 900 upon which various implementations may be implemented. For example, the computer system 900 may be implemented as the hemodynamic sensing system 100 (FIG. 1) in some implementations. Computer system 900 includes a bus 902 or other communication mechanism for communicating information, and a hardware processor, or multiple processors, 904 coupled with bus 902 for processing information. Hardware processor(s) 904 may be, for example, one or more general or special purpose microprocessors.

Computer system 900 also includes a main memory 906, such as a random access memory (RAM), cache and/or other dynamic storage devices, coupled to bus 902 for storing information and instructions to be executed by processor 904. Main memory 906 also may be used for storing temporary variables or other intermediate information during execution of instructions to be executed by processor 904. Such instructions, when stored in storage media accessible to processor 904, render computer system 900 into a special-purpose machine that is customized to perform the operations specified in the instructions.

Computer system 900 further includes a read only memory (ROM) 908 or other static storage device coupled to bus 902 for storing static information and instructions for processor 904. A storage device 910, such as a magnetic disk, optical disk, or USB thumb drive (Flash drive), etc., is provided and coupled to bus 902 for storing information and instructions.

Computer system 900 may be coupled via bus 902 to a display 912, such as a cathode ray tube (CRT) or LCD display (or touch screen), for displaying information to a computer user. An input device 914, including alphanumeric and other keys, is coupled to bus 902 for communicating information and command selections to processor 904. Another type of user input device is cursor control 916, such as a mouse, a trackball, or cursor direction keys for communicating direction information and command selections to processor 904 and for controlling cursor movement on display 912. This input device typically has two degrees of freedom in two axes, a first axis (e.g., x) and a second axis (e.g., y), that allows the device to specify positions in a plane. In some implementations, the same direction information and command selections as cursor control may be implemented via receiving touches on a touch screen without a cursor.

Computing system 900 may include a user interface module to implement a GUI that may be stored in a mass storage device as computer executable program instructions that are executed by the computing device(s). Computer system 900 may further, as described below, implement the techniques described herein using customized hard-wired logic, one or more ASICs or FPGAs, firmware and/or program logic which in combination with the computer system causes or programs computer system 900 to be a special-purpose machine. According to one implementation, the techniques herein are performed by computer system 900 in response to processor(s) 904 executing one or more sequences of one or more computer readable program instructions contained in main memory 906. Such instructions may be read into main memory 906 from another storage medium, such as storage device 910. Execution of the sequences of instructions contained in main memory 906 causes processor(s) 904 to perform the process steps described herein. In alternative implementations, hard-wired circuitry may be used in place of or in combination with software instructions.

Various forms of computer readable storage media may be involved in carrying one or more sequences of one or more computer readable program instructions to processor 904 for execution. For example, the instructions may initially be carried on a magnetic disk or solid state drive of a remote computer. The remote computer can load the instructions into its dynamic memory and send the instructions over a telephone line using a modem. A modem local to computer system 900 can receive the data on the telephone line and use an infra-red transmitter to convert the data to an infra-red signal. An infra-red detector can receive the data carried in the infra-red signal and appropriate circuitry can place the data on bus 902. Bus 902 carries the data to main memory 906, from which processor 904 retrieves and executes the instructions. The instructions received by main memory 906 may optionally be stored on storage device 910 either before or after execution by processor 904.

Computer system 900 also includes a communication interface 918 coupled to bus 902. Communication interface 918 provides a two-way data communication coupling to a network link 920 that is connected to a local network 922. For example, communication interface 918 may be an integrated services digital network (ISDN) card, cable modem, satellite modem, or a modem to provide a data communication connection to a corresponding type of telephone line. As another example, communication interface 918 may be a local area network (LAN) card to provide a data communication connection to a compatible LAN (or WAN component to communicated with a WAN). Wireless links may also be implemented. In any such implementation, communication interface 918 sends and receives electrical, electromagnetic or optical signals that carry digital data streams representing various types of information.

Network link 920 typically provides data communication through one or more networks to other data devices. For example, network link 920 may provide a connection through local network 922 to a host computer 924 or to data equipment operated by an Internet Service Provider (ISP) 926. ISP 926 in turn provides data communication services through the world wide packet data communication network now commonly referred to as the “Internet” 928. Local network 922 and Internet 928 both use electrical, electromagnetic or optical signals that carry digital data streams. The signals through the various networks and the signals on network link 920 and through communication interface 918, which carry the digital data to and from computer system 900, are example forms of transmission media.

Computer system 900 can send messages and receive data, including program code, through the network(s), network link 920 and communication interface 918. In the Internet example, a server 930 might transmit a requested code for an application program through Internet 928, ISP 926, local network 922 and communication interface 918.

The received code may be executed by processor 904 as it is received, and/or stored in storage device 910, or other non-volatile storage for later execution.

As described above, in various implementations certain functionality may be accessible by a user through a web-based viewer (such as a web browser), or other suitable software program). In such implementations, the user interface may be generated by a server computing system and transmitted to a web browser of the user (e.g., running on the user's computing system). Alternatively, data (e.g., user interface data) necessary for generating the user interface may be provided by the server computing system to the browser, where the user interface may be generated (e.g., the user interface data may be executed by a browser accessing a web service and may be configured to render the user interfaces based on the user interface data). The user may then interact with the user interface through the web-browser. User interfaces of certain implementations may be accessible through one or more dedicated software applications. In certain implementations, one or more of the computing devices and/or systems of the disclosure may include mobile computing devices, and user interfaces may be accessible through such mobile computing devices (for example, smartphones and/or tablets).

Many variations and modifications may be made to the above-described implementations, the elements of which are to be understood as being among other acceptable examples. All such modifications and variations are intended to be included herein within the scope of this disclosure. The foregoing description details certain implementations. It will be appreciated, however, that no matter how detailed the foregoing appears in text, the systems and methods can be practiced in many ways. As is also stated above, it should be noted that the use of particular terminology when describing certain features or aspects of the systems and methods should not be taken to imply that the terminology is being re-defined herein to be restricted to including any specific characteristics of the features or aspects of the systems and methods with which that terminology is associated.

Conditional language, such as, among others, “can,” “could,” “might,” or “may,” unless specifically stated otherwise, or otherwise understood within the context as used, is generally intended to convey that certain implementations include, while other implementations do not include, certain features, elements, and/or steps. Thus, such conditional language is not generally intended to imply that features, elements and/or steps are in any way required for one or more implementations or that one or more implementations necessarily include logic for deciding, with or without user input or prompting, whether these features, elements and/or steps are included or are to be performed in any particular implementation.

The term “substantially” when used in conjunction with the term “real-time” forms a phrase that will be readily understood by a person of ordinary skill in the art. For example, it is readily understood that such language will include speeds in which no or little delay or waiting is discernible, or where such delay is sufficiently short so as not to be disruptive, irritating, or otherwise vexing to a user.

Conjunctive language such as the phrase “at least one of X, Y, and Z,” or “at least one of X, Y, or Z,” unless specifically stated otherwise, is to be understood with the context as used in general to convey that an item, term, etc. may be either X, Y, or Z, or a combination thereof. For example, the term “or” is used in its inclusive sense (and not in its exclusive sense) so that when used, for example, to connect a list of elements, the term “or” means one, some, or all of the elements in the list. Thus, such conjunctive language is not generally intended to imply that certain implementations require at least one of X, at least one of Y, and at least one of Z to each be present.

The term “a” as used herein should be given an inclusive rather than exclusive interpretation. For example, unless specifically noted, the term “a” should not be understood to mean “exactly one” or “one and only one”; instead, the term “a” means “one or more” or “at least one,” whether used in the claims or elsewhere in the specification and regardless of uses of quantifiers such as “at least one,” “one or more,” or “a plurality” elsewhere in the claims or specification.

The term “comprising” as used herein should be given an inclusive rather than exclusive interpretation. For example, a computer comprising one or more processors should not be interpreted as excluding other computer components, and may possibly include such components as memory, input/output devices, and/or network interfaces, among others.

While the above detailed description has shown, described, and pointed out novel features as applied to various implementations, it may be understood that various omissions, substitutions, and changes in the form and details of the devices or processes illustrated may be made without departing from the spirit of the disclosure. As may be recognized, certain implementations of the inventions described herein may be embodied within a form that does not provide all of the features and benefits set forth herein, as some features may be used or practiced separately from others. Each of the disclosed aspects and examples of the present disclosure may be considered individually or in combination with other aspects, examples, and variations of the disclosure. The headings used herein are merely provided to enhance readability and are not intended to limit the scope of the embodiments disclosed in a particular section to the features or elements disclosed in that section. The features or elements from one embodiment of the disclosure can be employed by other embodiments of the disclosure. For example, features described in one figure may be used in conjunction with embodiments illustrated in other figures. The foregoing description and examples have been set forth merely to illustrate the disclosure and are not intended as being limiting. The scope of certain inventions disclosed herein is indicated by the appended claims rather than by the foregoing description. All changes which come within the meaning and range of equivalency of the claims are to be embraced within their scope.

Some example embodiments are included below for illustrative purposes. These examples should not be viewed as limiting.

In a 1st example, a hemodynamic sensor system configured to determine and display an endotype of hypotension of a patient, the system comprising: a hemodynamic sensor that produces a hemodynamic sensor signal representative of an arterial pressure signal waveform of the patient; a user interface; a non-transitory memory having executable instructions stored thereon; and an electronic hardware processor in communication with the non-transitory memory and configured to execute the instructions to cause the system to at least: receive, from the hemodynamic sensor, the hemodynamic sensor signal from the patient; determine, based on the arterial pressure signal waveform, the endotype of hypotension of the patient; and generate, based on the determined endotype of hypotension of the patient, data for displaying on the user interface an alert indicating the endotype of hypotension of the patient.

In a 2nd example, the hemodynamic sensor system of Example 1, wherein determining the endotype of hypotension of the patient comprises: extracting from the arterial pressure signal waveform a plurality of heart health parameters; encoding, using a deep learning model, the plurality of heart health parameters into one or more latent space heart health parameters; and generating a location in latent space of the arterial pressure signal waveform using the one or more latent space heart health parameters.

In a 3rd example, the hemodynamic sensor system of Example 2, wherein determining the endotype of hypotension of the patient further comprises: determining, based on the location of the arterial pressure signal waveform in latent space, a relative location of the arterial pressure signal waveform in latent space based on a set of clusters from reference arterial pressure signal waveforms within the latent space; determining, based on the set of clusters, a cluster associated with the arterial pressure signal waveform in latent space; and based on the cluster of the arterial pressure signal waveform, determine the endotype of hypotension of the patient.

In a 4th example, the hemodynamic sensor system of any of Examples 2-3, wherein the alert includes a graphical user interface feature that indicates at least one of the plurality of heart health parameters that characterize the endotype of hypotension of the patient.

In a 5th example, the hemodynamic sensor system of Example 4, wherein the at least one of the plurality of heart health parameters includes a stroke volume index (SVI), a heart rate (HR), a cardiac index (CI), a systemic vascular resistance index (SVRI), and a stroke volume variation (SVV).

In a 6th example, the hemodynamic sensor system of any of Examples 1-5, wherein the endotype of hypotension is selected from the following group: a vasodilation endotype, a myocardial depression endotype, a bradycardia endotype, and a hypovolemia endotype.

In a 7th example, the hemodynamic sensor system of Example 1, wherein a plurality of heart health parameters are extracted from the arterial pressure signal waveform, wherein the heart health parameters are encoded into a latent space to form latent space heart health parameters, wherein generating data for display comprises causing presentation of the user interface, and wherein the user interface: presents a plurality of clusters, wherein individual clusters are associated with individual endotypes, and wherein each cluster is formed from latent space heart health parameters associated with respective patients, and updates based on information indicating a particular patient and presents an endotype trend associated with the particular patient.

In a 8th example, the hemodynamic sensor system of Example 7, wherein one or more probability areas are included for each cluster, and wherein each probability area indicates a likelihood of a corresponding endotype associated with the cluster.

In a 9th example, the hemodynamic sensor system of Example 7, wherein the clusters are presented as being different colors.

In a 10th example, the hemodynamic sensor system of Example 7, wherein the endotype trend is presented as a plurality of graphical elements.

In a 11th example, the hemodynamic sensor system of Example 10, wherein the graphical elements are adjusted based on a recency in time of the latent space heart health parameters.

In a 12th example, the hemodynamic sensor system of Example 11, wherein an interior color of each graphical element is adjusted in transparency based on its recency in time.

In a 13th example, the hemodynamic sensor system of Example 7, wherein the endotype trend for the particular patient is a trendline.

In a 14th example, the hemodynamic sensor system of any of Examples 1-13, wherein: the hemodynamic sensor signal comprises an analog signal and the system comprises an analog-to-digital converter that converts the hemodynamic sensor signal to the arterial pressure signal waveform; and determining the endotype of hypotension of the patient includes converting, using the analog-to-digital converter, the hemodynamic sensor signal to the arterial pressure signal waveform.

In a 15th example, a hemodynamic sensor system configured to determine and display an endotype of hypotension of a patient, the system comprising: a hemodynamic sensor that produces a hemodynamic sensor signal representative of an arterial pressure signal waveform of the patient; an infusion pump; a non-transitory memory having executable instructions stored thereon; and an electronic hardware processor in communication with the non-transitory memory and configured to execute the instructions to cause the system to at least: receive, from the hemodynamic sensor, the hemodynamic sensor signal from the patient; determine, based on the arterial pressure signal waveform, the endotype of hypotension of the patient; and generate a control signal for the infusion pump to deliver an intravenous therapy to the patient based on the determined endotype of hypotension of the patient and a determined therapeutic protocol for the patient.

In a 16th example, the hemodynamic sensor system of Example 15, wherein determining the endotype of hypotension of the patient comprises: extracting from the arterial pressure signal waveform a plurality of heart health parameters; encoding, using a deep learning model, the plurality of heart health parameters into one or more latent space heart health parameters; and generating a location in latent space of the arterial pressure signal waveform using the one or more latent space heart health parameters.

In a 17th example, the hemodynamic sensor system of Example 16, wherein determining the endotype of hypotension of the patient further comprises: determining, based on the location of the arterial pressure signal waveform in latent space, a relative location of the arterial pressure signal waveform in latent space based on a set of clusters from reference arterial pressure signal waveforms within the latent space; determining, based on the set of clusters, a cluster associated with the arterial pressure signal waveform in latent space; and determining, based on the cluster of the arterial pressure signal waveform, the endotype of hypotension of the patient.

In an 18th example, the hemodynamic sensor system of Example 17, wherein executing the instructions further cause the system to: generate an alert based on the determined endotype of hypotension of the patient.

In a 19th example, the hemodynamic sensor system of Example 18, further comprising a graphical user interface feature that indicates the alert and at least one of the plurality of heart health parameters that characterize the endotype of hypotension of the patient.

In a 20th example, the hemodynamic sensor system of Examples 18 or 19, wherein delivering the intravenous therapy to the patient using the infusion pump requires approval by a user of the system.

In a 21st example, the hemodynamic sensor system of any of Example 16-20, wherein the at least one of the plurality of heart health parameters includes a stroke volume index (SVI), a heart rate (HR), a cardiac index (CI), a systemic vascular resistance index (SVRI), and a stroke volume variation (SVV).

In a 22nd example, the hemodynamic sensor system of any of Examples 15-21, wherein the endotype of hypotension is selected from the following group: a vasodilation endotype, a myocardial depression endotype, a bradycardia endotype, and a hypovolemia endotype.

In a 23rd example, the hemodynamic sensor system of Example 19, wherein a plurality of heart health parameters are extracted from the arterial pressure signal waveform, wherein the heart health parameters are encoded into a latent space to form latent space heart health parameters, wherein generating data for display comprises causing presentation of the user interface, and wherein the user interface: presents a plurality of clusters, wherein individual clusters are associated with individual endotypes, and wherein each cluster is formed from latent space heart health parameters associated with respective patients; and updates based on information indicating a particular patient and presents an endotype trend associated with the particular patient.

In a 24th example, the hemodynamic sensor system of Example 23, wherein one or more probability areas are included for each cluster, and wherein each probability area indicates a likelihood of a corresponding endotype associated with the cluster.

In a 25th example, the hemodynamic sensor system of Example 23, wherein the endotype trend is presented as a plurality of graphical elements.

In a 26th example, the hemodynamic sensor system of Example 23, wherein the endotype trend for the particular patient is a trendline.

In a 27th example, the hemodynamic sensor system of any of Examples 15-26, wherein: the hemodynamic sensor signal comprises an analog signal and the system comprises an analog-to-digital converter that converts the hemodynamic sensor signal to the arterial pressure signal waveform; and determining the endotype of hypotension of the patient includes converting, using the analog-to-digital converter, the hemodynamic sensor signal to the arterial pressure signal waveform.

In a 28th example, the hemodynamic sensor system of Example 15, wherein determining the endotype of hypotension of the patient comprises: extracting from the arterial pressure signal waveform a plurality of heart health parameters; comparing the plurality of heart health parameters with a reference that describes a profile of each of the endotype clusters for each of the plurality of heart health parameters; and determining the endotype of hypotension of the patient based on a best-fit model of the plurality of heart health parameters with the clusters.

In a 29th example, the system of Example 28 wherein the clusters associated with the arterial pressure signal are formed in latent space and then used to generate physical parameters.

In a 30th example, a hemodynamic sensor system configured to determine an endotype of hypotension of a patient, the system comprising: a hemodynamic sensor that produces an analog hemodynamic sensor signal representative of an arterial pressure signal waveform of the patient; an analog-to-digital converter that converts the analog hemodynamic sensor signal to the arterial pressure signal waveform; a non-transitory memory having executable instructions and a fully connected deep learning model stored thereon; and an electronic hardware processor in communication with the non-transitory memory and configured to execute the instructions to cause the system to at least: receive, from the hemodynamic sensor, the analog hemodynamic sensor signal from the patient; convert, using the analog-to-digital converter, the analog hemodynamic sensor signal to the arterial pressure signal waveform; extract from the arterial pressure signal waveform a plurality of heart health parameters encode, using the fully connected deep learning model, the plurality of heart health parameters into one or more latent space heart health parameters; generate a location in latent space of the arterial pressure signal waveform using the one or more latent space heart health parameters; determine, based on the location of the arterial pressure signal waveform in latent space, a relative location of the arterial pressure signal waveform in latent space, wherein determining the relative location of the arterial pressure signal waveform in latent space comprises: obtaining a plurality of reference arterial pressure signal waveforms from a plurality of patients; extracting from the plurality of reference arterial pressure signal waveforms a plurality of reference sets of heart health parameters; combining each of the plurality of reference sets of heart health parameters into a plurality of corresponding one or more reference latent space heart health parameters; generating reference locations in latent space of each of the plurality of reference arterial pressure signal waveforms based on the plurality of one or more reference latent space heart health parameters of each of the plurality of reference sets of heart health; determining a clustering evaluation metric of the reference locations in latent space, the clustering evaluation metric configured to indicate a goodness of clustering of the reference locations; based on the clustering evaluation metric, determining a best number of clusters associated with the reference locations; associating, based on the best number of clusters, each of the reference arterial pressure signal waveforms to a corresponding cluster in latent space to determine a set of clusters; and determining, based on the set of clusters, a cluster associated with the arterial pressure signal waveform in latent space; based on the cluster of the arterial pressure signal waveform, determine the endotype of hypotension of the patient; and generate, based on the determined endotype of hypotension of the patient, data for displaying an alert indicating the endotype of hypotension of the patient.

In a 31st example, the hemodynamic sensor system of Example 30, wherein the electronic hardware processor is further configured to execute the instructions to cause the system to at least: decode, using the fully connected deep learning model, the one or more latent space heart health parameters into the plurality of heart health parameters in original space.

In a 32nd example, the hemodynamic sensor system of Example 31, wherein the electronic hardware processor is further configured to execute the instructions to cause the system to at least: determine, based on the decoded plurality of heart health parameters in original space, reference locations in original space of each of the plurality of reference arterial pressure signal waveforms; and validate the determined best number of clusters associated with the reference locations by comparing the determined reference locations in latent space with the determined reference locations in original space.

In a 33rd example, the hemodynamic sensor system of any of Examples 30-32, wherein the plurality of heart health parameters comprises one or more of: a first heart health parameter comprising a stroke volume index (SVI); a second heart health parameter comprising a heart rate (HR); a third heart health parameter comprising a cardiac index (CI); a fourth heart health parameter comprising a systemic vascular resistance index (SVRI); and a fifth heart health parameter comprising a stroke volume variation (SVV).

In a 34th example, the hemodynamic sensor system of Example 33, wherein the electronic hardware processor is further configured to execute the instructions to cause the system to at least: generate, based on the determined endotype of hypotension of the patient, data for displaying at least one of the first, second, third, fourth, or fifth heart health parameters.

In a 35th example, the hemodynamic sensor system of any of Examples 30-34, wherein the clustering evaluation metric comprises at least one of: a silhouette metric, a Calinski-Harabasz index, a Davies-Bouldin Index, or a Gaussian Mixture Model.

In a 36th example, the hemodynamic sensor system of any of Examples 30-35, wherein the fully connected deep learning model comprises an autoencoder configured to automatically convert the plurality of heart health parameters into the one or more latent space heart health parameters.

In a 37th example, the hemodynamic sensor system of any of Examples 30-36, wherein the relative location of the arterial pressure signal waveform in latent space comprises the cluster associated with the arterial pressure signal waveform in latent space.

In a 38th example, the hemodynamic sensor system of any of Examples 30-37, wherein the endotype of hypotension comprises at least one of: a vasodilation endotype, a myocardial depression endotype, a bradycardia endotype, or a hypovolemia endotype.

In a 39th example, the hemodynamic sensor system of any of Examples 30-38, wherein the best number of clusters associated with the reference locations is exactly four clusters.

In a 40th example, a hemodynamic sensor system configured to determine an endotype of hypotension of a patient, the system comprising: a hemodynamic sensor that produces an analog hemodynamic sensor signal representative of an arterial pressure signal waveform of the patient; an analog-to-digital converter that converts the analog hemodynamic sensor signal to the arterial pressure signal waveform; a non-transitory memory having executable instructions and a fully connected deep learning model stored thereon; and an electronic hardware processor in communication with the non-transitory memory and configured to execute the instructions to cause the system to at least: receive, from the hemodynamic sensor, the analog hemodynamic sensor signal from the patient; convert, using the analog-to-digital converter, the analog hemodynamic sensor signal to the arterial pressure signal waveform; extract from the arterial pressure signal waveform a plurality of heart health parameters; encode, using the fully connected deep learning model, the plurality of heart health parameters into one or more latent space heart health parameters; generate a location in latent space of the arterial pressure signal waveform using the one or more latent space heart health parameters; determine, based on the location of the arterial pressure signal waveform in latent space, a relative location of the arterial pressure signal waveform in latent space, wherein determining the relative location of the arterial pressure signal waveform in latent space comprises: determining a set of clusters from a plurality of reference arterial pressure signal waveforms; and determining, based on the set of clusters, a cluster associated with the arterial pressure signal waveform in latent space; based on the cluster of the arterial pressure signal waveform, determine the endotype of hypotension of the patient; and generate, based on the determined endotype of hypotension of the patient, data for displaying an alert indicating the endotype of hypotension of the patient.

In a 41st example, the hemodynamic sensor system of Example 40, wherein the electronic hardware processor is further configured to execute the instructions to cause the system to at least: decode, using the fully connected deep learning model, the one or more latent space heart health parameters into the plurality of heart health parameters in original space.

In a 42nd example, the hemodynamic sensor system of Example 41, wherein the electronic hardware processor is further configured to execute the instructions to cause the system to at least: determine, based on the decoded plurality of heart health parameters in original space, reference locations in original space of each of the plurality of reference arterial pressure signal waveforms; and validate the determined cluster associated with the arterial pressure signal waveform in latent space by comparing the determined reference locations in latent space with the determined reference locations in original space.

In a 43rd example, the hemodynamic sensor system of any of Examples 40 to 42, wherein the plurality of heart health parameters comprises one or more of: a first heart health parameter comprising a stroke volume index (SVI); a second heart health parameter comprising a heart rate (HR); a third heart health parameter comprising a cardiac index (CI); a fourth heart health parameter comprising a systemic vascular resistance index (SVRI); and a fifth heart health parameter comprising a stroke volume variation (SVV).

In a 44th example, the hemodynamic sensor system of Example 43, wherein the electronic hardware processor is further configured to execute the instructions to cause the system to at least: generate, based on the determined endotype of hypotension of the patient, data for displaying at least one of the first, second, third, fourth, or fifth heart health parameters.

In a 45th example, the hemodynamic sensor system of any of Examples 40 to 44 wherein determining the set of clusters from the reference arterial pressure signal waveforms comprises: determining a clustering evaluation metric of the reference arterial pressure signal waveforms, the clustering evaluation metric configured to indicate a goodness of clustering of the reference locations; and based on the clustering evaluation metric, determining a best number of clusters associated with the reference locations.

In a 46th example, the hemodynamic sensor system of Example 45, wherein the clustering evaluation metric comprises at least one of: a silhouette metric, a Calinski-Harabasz index, a Davies-Bouldin Index, or a Gaussian Mixture Model.

In a 47th example, the hemodynamic sensor system of Example 45, wherein the best number of clusters associated with the reference locations is exactly four clusters.

In a 48th example, the hemodynamic sensor system of any of Examples 40 to 47, wherein the fully connected deep learning model comprises an autoencoder configured to automatically convert the plurality of heart health parameters into the one or more latent space heart health parameters.

In a 49th example, the hemodynamic sensor system of any of Examples 40 to 48, wherein the relative location of the arterial pressure signal waveform in latent space comprises the cluster associated with the arterial pressure signal waveform in latent space.

In a 50th example, the hemodynamic sensor system of any of Examples 40 to 49, wherein the endotype of hypotension comprises at least one of: a vasodilation endotype, a myocardial depression endotype, a bradycardia endotype, or a hypovolemia endotype.

In a 51st example, a hemodynamic sensor system configured to generate a trained model for determining an endotype of hypotension of a patient, the system comprising: a hemodynamic sensor that produces analog hemodynamic sensor signals representative of arterial pressure signal waveforms of patients; an analog-to-digital converter that converts the analog hemodynamic sensor signals to the arterial pressure signal waveforms; a non-transitory memory having executable instructions and a fully connected deep learning model stored thereon; and an electronic hardware processor in communication with the non-transitory memory and configured to execute the instructions to cause the system to at least: receive, from the hemodynamic sensor, a plurality of analog hemodynamic sensor signals from a plurality of reference patients; convert, using the analog-to-digital converter, the plurality of reference analog hemodynamic sensor signals to a corresponding plurality of reference arterial pressure signal waveforms; extract from the plurality of reference arterial pressure signal waveforms corresponding sets of reference heart health parameters; encode, using the fully connected deep learning model, the plurality of reference sets of heart health parameters into a plurality of corresponding one or more reference latent space heart health parameters; generate reference locations in latent space of each of the plurality of reference arterial pressure signal waveforms based on the plurality of one or more reference latent space heart health parameters of each of the plurality of reference sets of heart health; determine a clustering evaluation metric of the reference locations in latent space, the clustering evaluation metric configured to indicate a goodness of clustering of the reference locations; based on the clustering evaluation metric, determine a best number of clusters associated with the reference locations; associate, based on the best number of clusters, each of the reference arterial pressure signal waveforms to a corresponding cluster in latent space to determine a set of clusters; determine, based on the set of clusters and on the reference locations in latent space of each of the plurality of reference arterial pressure signal waveforms, a cluster associated with each of the reference arterial pressure signal waveform in latent space; based on the cluster associated with each of the reference arterial pressure signal waveforms, determine the endotype of hypotension of each of the reference patients; decode, using the fully connected deep learning model, the plurality of one or more reference latent space heart health parameters into the corresponding plurality of reference sets of heart health parameters; and generate a trained model for determining the endotype of hypotension of the patient.

In a 52nd example, the hemodynamic sensor system of Example 51, wherein each of the sets of the plurality of reference heart health parameters comprises one or more of: a first heart health parameter comprising a stroke volume index (SVI); a second heart health parameter comprising a heart rate (HR); a third heart health parameter comprising a cardiac index (CI); a fourth heart health parameter comprising a systemic vascular resistance index (SVRI); and a fifth heart health parameter comprising a stroke volume variation (SVV).

In a 53rd example, the hemodynamic sensor system of Example 52, wherein the electronic hardware processor is further configured to execute the instructions to cause the system to at least: generate, based on the determined endotype of hypotension of the patient, data for displaying at least one of the first, second, third, fourth, or fifth heart health parameters.

In a 54th example, the hemodynamic sensor system of any of Examples 36 to 38, wherein the clustering evaluation metric comprises at least one of: a silhouette metric, a Calinski-Harabasz index, a Davies-Bouldin Index, or a Gaussian Mixture Model.

In a 55th example, the hemodynamic sensor system of any of Examples 51 to 54, wherein the best number of clusters associated with the reference locations is exactly four clusters.

In a 56th example, the hemodynamic sensor system of any of Examples 51 to 55, wherein the fully connected deep learning model comprises an autoencoder configured to automatically convert the plurality of heart health parameters into the one or more latent space heart health parameters.

In a 57th example, the hemodynamic sensor system of any of Examples 51 to 56, wherein the endotype of hypotension comprises at least one of: a vasodilation endotype, a myocardial depression endotype, a bradycardia endotype, or a hypovolemia endotype.

In a 58th example, a hemodynamic sensor system configured to determine an endotype of hypotension of a patient, the system comprising: a hemodynamic sensor that produces an analog hemodynamic sensor signal representative of an arterial pressure signal waveform of the patient; an analog-to-digital converter that converts the analog hemodynamic sensor signal to the arterial pressure signal waveform; a non-transitory memory having executable instructions and a fully connected deep learning model stored thereon; and an electronic hardware processor in communication with the non-transitory memory and configured to execute the instructions to cause the system to at least: receive, from the hemodynamic sensor, the analog hemodynamic sensor signal from the patient; convert, using the analog-to-digital converter, the analog hemodynamic sensor signal to the arterial pressure signal waveform; extract from the arterial pressure signal waveform a plurality of heart health parameters comprising: a first heart health parameter comprising a stroke volume index (SVI); a second heart health parameter comprising a heart rate (HR); a third heart health parameter comprising a cardiac index (CI); a fourth heart health parameter comprising a systemic vascular resistance index (SVRI); and a fifth heart health parameter comprising a stroke volume variation (SVV); encode, using the fully connected deep learning model, the plurality of heart health parameters into first and second latent space heart health parameters; generate a location in latent space of the arterial pressure signal waveform using the first and second latent space heart health parameters; determine, based on the location of the arterial pressure signal waveform in latent space, a relative location of the arterial pressure signal waveform in latent space, wherein determining the relative location of the arterial pressure signal waveform in latent space comprises: obtaining a plurality of reference arterial pressure signal waveforms from a plurality of patients; extracting from the plurality of reference arterial pressure signal waveforms a plurality of reference sets of heart health parameters, each of the reference sets of heart health parameters comprising a corresponding SVI, HR, CI, SVRI, and SVV; combining each of the plurality of reference sets of heart health parameters into a plurality of corresponding first and second reference latent space heart health parameters; generating reference locations in latent space of each of the plurality of reference arterial pressure signal waveforms based on the plurality of first and second reference latent space heart health parameters of each of the plurality of reference sets of heart health; determining a silhouette criteria of the reference locations in latent space, the silhouette criteria configured to indicate a goodness of clustering of the reference locations; based on the silhouette criteria, determining a best number of clusters associated with the reference locations; associating, based on the best number of clusters, each of the reference arterial pressure signal waveforms to a corresponding cluster in latent space to determine a set of clusters; and determining, based on the set of clusters, a cluster associated with the arterial pressure signal waveform in latent space; based on the cluster of the arterial pressure signal waveform, determine the endotype of hypotension of the patient; and generate, based on the determined endotype of hypotension of the patient, data for displaying an alert indicating the endotype of hypotension of the patient and at least one of the first, second, third, fourth, or fifth heart health parameters.

In a 59th example, the hemodynamic sensor system of Example 58, wherein the electronic hardware processor is further configured to execute the instructions to cause the system to at least: decode, using the fully connected deep learning model, the first and second latent space heart health parameters into the plurality of heart health parameters.

In a 60th example, a hemodynamic sensor system configured to determine an endotype of hypotension of a patient, the system comprising: a hemodynamic sensor that produces an analog hemodynamic sensor signal representative of an arterial pressure signal waveform of the patient; an analog-to-digital converter that converts the analog hemodynamic sensor signal to the arterial pressure signal waveform; a non-transitory memory having executable instructions and a fully connected deep learning model stored thereon; and an electronic hardware processor in communication with the non-transitory memory and configured to execute the instructions to cause the system to at least: receive, from the hemodynamic sensor, the analog hemodynamic sensor signal from the patient; convert, using the analog-to-digital converter, the analog hemodynamic sensor signal to the arterial pressure signal waveform; extract from the arterial pressure signal waveform a plurality of heart health parameters comprising: a first heart health parameter comprising a stroke volume index (SVI); a second heart health parameter comprising a heart rate (HR); a third heart health parameter comprising a cardiac index (CI); a fourth heart health parameter comprising a systemic vascular resistance index (SVRI); and a fifth heart health parameter comprising a stroke volume variation (SVV); encode, using the fully connected deep learning model, the plurality of heart health parameters into first and second latent space heart health parameters; generate a location in latent space of the arterial pressure signal waveform using the first and second latent space heart health parameters; determine, based on the location of the arterial pressure signal waveform in latent space, a relative location of the arterial pressure signal waveform in latent space, wherein determining the relative location of the arterial pressure signal waveform in latent space comprises: determining a set of clusters from reference arterial pressure signal waveforms; and determining, based on the set of clusters, a cluster associated with the arterial pressure signal waveform in latent space; based on the cluster of the arterial pressure signal waveform, determine the endotype of hypotension of the patient; and generate, based on the determined endotype of hypotension of the patient, data for displaying an alert indicating the endotype of hypotension of the patient and at least one of the first, second, third, fourth, or fifth heart health parameters.

In a 61st example, a hemodynamic sensor system configured to generate a trained model for determining an endotype of hypotension of a patient, the system comprising: a hemodynamic sensor that produces analog hemodynamic sensor signals representative of arterial pressure signal waveforms of patients; an analog-to-digital converter that converts the analog hemodynamic sensor signals to the arterial pressure signal waveforms; a non-transitory memory having executable instructions and a fully connected deep learning model stored thereon; and an electronic hardware processor in communication with the non-transitory memory and configured to execute the instructions to cause the system to at least: receiving, from the hemodynamic sensor, a plurality of analog hemodynamic sensor signals from a plurality of reference patients; convert, using the analog-to-digital converter, the plurality of reference analog hemodynamic sensor signals to a corresponding plurality of reference arterial pressure signal waveforms; extract from the plurality of reference arterial pressure signal waveforms corresponding sets of reference heart health parameters, each of the sets of reference heart health parameters comprising: a first heart health parameter comprising a stroke volume index (SVI); a second heart health parameter comprising a heart rate (HR); a third heart health parameter comprising a cardiac index (CI); a fourth heart health parameter comprising a systemic vascular resistance index (SVRI); and a fifth heart health parameter comprising a stroke volume variation (SVV); encode, using the fully connected deep learning model, the plurality of reference sets of heart health parameters into a plurality of corresponding first and second reference latent space heart health parameters; generate reference locations in latent space of each of the plurality of reference arterial pressure signal waveforms based on the plurality of first and second reference latent space heart health parameters of each of the plurality of reference sets of heart health; determine a silhouette criteria of the reference locations in latent space, the silhouette criteria configured to indicate a goodness of clustering of the reference locations; based on the silhouette criteria, determine a best number of clusters associated with the reference locations; associate, based on the best number of clusters, each of the reference arterial pressure signal waveforms to a corresponding cluster in latent space to determine a set of clusters; determine, based on the set of clusters and on the reference locations in latent space of each of the plurality of reference arterial pressure signal waveforms, a cluster associated with each of the reference arterial pressure signal waveform in latent space; based on the cluster associated with each of the reference arterial pressure signal waveforms, determine the endotype of hypotension of each of the reference patients; decode, using the fully connected deep learning model, the plurality of first and second reference latent space heart health parameters into the corresponding plurality of reference sets of heart health parameters; and generate a trained model for determining the endotype of hypotension of the patient.

In a 62nd example, a method implemented by a system of one or more processors, wherein the system generates a user interface for presentation, and wherein the user interface: presents a plurality of data point clusters, wherein individual data point clusters are associated with individual endotypes, and wherein each data point cluster is formed from data points associated with a plurality of patients, and updates based on information indicating a particular patient and presents an endotype trend reflecting a plurality of data points associated with the particular patient.

In a 63rd example, the method of Example 62, wherein the particular patient is not included in the plurality of patients.

In a 64th example, the method of Example 62, wherein the data points are clustered based on latent space heart health parameters determined for each patient at different points in time.

In a 65th example, the method of Example 62, wherein the particular patient is not included in the plurality of patients, wherein latent space heart health parameters are determined for the particular patient at different times, and wherein the latent space heart health parameters are mapped to the data point clusters.

In a 66th example, the method of Example 62, wherein the data point clusters are presented as being different colors.

In a 67th example, the method of Example 62, wherein the endotype trend is presented as a plurality of graphical elements associated with the plurality of data points associated with the particular patient.

In a 68th example, the method of Example 67, wherein the graphical elements are stars.

In a 69th example, the method of Example 67, wherein the graphical elements are adjusted based on a recency of the data point.

In a 70th example, the method of Example 69, wherein an interior color of each graphical element is adjusted in transparency based on its recency.

In a 71st example, the method of Example 70, wherein the user interface presents a plurality of ellipses, and wherein each ellipse includes a subset of the data points which are assigned similar likelihoods of a particular endotype.

In a 72nd example, the method of Example 70, wherein the data point clusters are assigned a same color.

In a 73rd example, the method of Example 70, wherein the endotype trend is a trendline connecting the data points associated with the particular patient.

In a 74th example, the method of Example 70, wherein a textual summary of the endotype trend is included in the user interface.

In a 75th example, the system comprising one or more processors and compute storage media storing instructions that when executed by the one or more processors, cause the processors to perform the method of Examples 62-74.

In a 76th example, non-transitory computer storage media storing instructions that when executed by a system of one or more processors, cause the one or more processors to perform the method of Examples 62-74.

In a 77th example, a computer-implemented method comprising: using input data from a database reflecting latent feature space heart health parameters computed previously for a plurality of patients, the input data including likelihoods associated with endotypes of hypotension for the patients, and the input data being associated with a plurality of times; and generating a user interface for presentation via a user device, wherein the user interface: presents a plurality of data point clusters computed previously from the database, wherein individual data point clusters are associated with individual endotypes, and wherein each data point cluster is formed from data points included in the input data, and presents a plurality of data points obtained in real time for a new and currently monitored patient not being part of the database with data points of a plurality of patients, and presents an endotype trend over time reflecting a plurality of data points associated with the new and currently monitored patient, the data points being associated with at least two or more times.

In a 78th example, the method of Example 77, wherein the user interface presents a plurality of ellipses for each cluster of datapoints from the database, and wherein each ellipse includes a subset of the data points which are assigned similar likelihoods of a particular endotype within each cluster of datapoints from the database.

In a 79th example, the method of Example 77-78, wherein the data point clusters are presented as being different colors.

In an 80th example, the method of Example 77-79, wherein the endotype trend is presented as a plurality of graphical elements associated with the plurality of data points associated with the new and currently monitored patient.

In an 81st example, the method of Example 80, wherein the graphical elements are stars.

In an 82nd example, the method of Example 81, wherein the graphical elements are adjusted based on a recency in time of the data point.

In an 83rd example, the method of any of Examples 77-82, wherein an interior color of each graphical element is adjusted in transparency based on its recency in time.

In an 84th example, the method of any of Examples 77-83, wherein the data point clusters from the database are assigned a same color.

In an 85th example, the method of any of Examples 77-84, wherein the endotype trend for the new and currently monitored patient is a trendline connecting the data points associated with the new and currently monitored patient.

In an 86th example, the method of any of Examples 77-85, wherein a textual summary of the endotype clusters for the database and the endotype trend for the new and currently monitored patient is included in the user interface.

In an 87th example, a system comprising one or more processors and compute storage media storing instructions that when executed by the one or more processors, cause the processors to perform the method of any of Examples 77-86.

In an 88th example, the non-transitory computer storage media storing instructions that when executed by a system of one or more processors, cause the one or more processors to perform the method of any of Examples 77-87.

In an 89th example, the hemodynamic sensor system of Example 3, wherein generating the location in latent space of the arterial pressure signal waveform using the one or more latent space heart health parameters comprises: determining, over a plurality of time points, a plurality of locations in the latent space of a plurality of arterial pressure signal waveforms using corresponding latent space heart health parameters.

In a 90th example, the hemodynamic sensor system of Example 89, wherein determining the plurality of locations in latent space of the plurality of arterial pressure signal waveforms using the corresponding latent space heart health parameters comprises: determining a harmonic probability associated with the plurality of locations in latent space of the plurality of arterial pressure signal waveforms.

In a 91st example, the hemodynamic sensor system of Example 89, wherein determining the plurality of locations in latent space of the plurality of arterial pressure signal waveforms using the corresponding latent space heart health parameters comprises: determining a Gaussian distribution associated with the plurality of locations in latent space of the plurality of arterial pressure signal waveforms.

In a 92nd example, the hemodynamic sensor system of Example 89, wherein the alert comprises a plurality of indicators corresponding to each of the plurality of time points.

In a 93rd example, the hemodynamic sensor system of Example 92, wherein at least one of the plurality of indicators is indicative of a time point relative to at least one of the other of the plurality of indicators.

In a 94th example, the hemodynamic sensor system of Example 1, wherein determining the endotype of hypotension of the patient comprises: determining a hypotension probability index (HPI) corresponding to the probability of the patient developing the endotype of hypotension.

In a 95th example, the hemodynamic sensor system of Example 94, wherein generating the data for displaying an alert indicating the endotype of hypotension of the patient is further based on the HPI being above a threshold HPI.

In a 96th example, the hemodynamic sensor system of Example 1, wherein the processor is configured to execute the instructions to further cause the system to at least: determine, based on the determined endotype of hypotension, a therapy protocol for the patient.

In a 97th example, the hemodynamic sensor system of Example 96, wherein the processor is configured to execute the instructions to further cause the system to at least: generate, based on the determined therapy protocol, a command configured to cause an infusion pump to deliver an intravenous therapeutic agent to the patient.

In a 98th example, the hemodynamic sensor system of Example 97, further comprising the infusion pump.

In a 99th example, the hemodynamic sensor system of Example 1, wherein determining the endotype of hypotension of the patient comprises: extracting from the arterial pressure signal waveform a plurality of heart health parameters; comparing the plurality of heart health parameters with a reference that describes a profile of each of the endotype clusters for each of the plurality of heart health parameters; and determining the endotype of hypotension of the patient based on a best-fit model of the plurality of heart health parameters with the clusters.

In a 100th example, the hemodynamic sensor system of Example 99, wherein the reference comprises a table.

What is claimed is:

1. A hemodynamic sensor system configured to determine and display an endotype of hypotension of a patient, the system comprising:
a hemodynamic sensor that produces a hemodynamic sensor signal representative of an arterial pressure signal waveform of the patient;
an infusion pump;
a non-transitory memory having executable instructions stored thereon; and
an electronic hardware processor in communication with the non-transitory memory and configured to execute the instructions to cause the system to at least:
receive, from the hemodynamic sensor, the hemodynamic sensor signal from the patient;
determine, based on the arterial pressure signal waveform, the endotype of hypotension of the patient; and
generate a control signal for the infusion pump to deliver an intravenous therapy to the patient based on the determined endotype of hypotension of the patient and a determined therapeutic protocol for the patient.

2. The hemodynamic sensor system of claim 1, wherein determining the endotype of hypotension of the patient comprises:
extracting from the arterial pressure signal waveform a plurality of heart health parameters;
encoding, using a deep learning model, the plurality of heart health parameters into one or more latent space heart health parameters; and
generating a location in latent space of the arterial pressure signal waveform using the one or more latent space heart health parameters.

3. The hemodynamic sensor system of claim 2, wherein determining the endotype of hypotension of the patient further comprises:
determining, based on the location of the arterial pressure signal waveform in latent space, a relative location of the arterial pressure signal waveform in latent space based on a set of clusters from reference arterial pressure signal waveforms within the latent space;
determining, based on the set of clusters, a cluster associated with the arterial pressure signal waveform in latent space; and
determining, based on the cluster of the arterial pressure signal waveform, the endotype of hypotension of the patient.

4. The hemodynamic sensor system of claim 3, wherein executing the instructions further cause the system to:
generate an alert based on the determined endotype of hypotension of the patient.

5. The hemodynamic sensor system of claim 4, further comprising a graphical user interface feature that indicates the alert and at least one of the plurality of heart health parameters that characterize the endotype of hypotension of the patient.

6. The hemodynamic sensor system of claim 4, wherein delivering the intravenous therapy to the patient using the infusion pump requires approval by a user of the system.

7. The hemodynamic sensor system of claim 2, wherein the at least one of the plurality of heart health parameters includes a stroke volume index (SVI), a heart rate (HR), a cardiac index (CI), a systemic vascular resistance index (SVRI), and a stroke volume variation (SVV).

8. The hemodynamic sensor system of claim 1, wherein the endotype of hypotension is selected from the following group: a vasodilation endotype, a myocardial depression endotype, a bradycardia endotype, and a hypovolemia endotype.

9. The hemodynamic sensor system of claim 5, wherein a plurality of heart health parameters are extracted from the arterial pressure signal waveform, wherein the heart health parameters are encoded into a latent space to form latent space heart health parameters, wherein generating data for display comprises causing presentation of the user interface, and wherein the user interface:
presents a plurality of clusters, wherein individual clusters are associated with individual endotypes, and wherein each cluster is formed from latent space heart health parameters associated with respective patients; and
updates based on information indicating a particular patient and presents an endotype trend associated with the particular patient.

10. The hemodynamic sensor system of claim 9, wherein one or more probability areas are included for each cluster, and wherein each probability area indicates a likelihood of a corresponding endotype associated with the cluster.

11. The hemodynamic sensor system of claim 10, wherein the endotype trend is presented as a plurality of graphical elements.

12. The hemodynamic sensor system of claim 1, wherein:
the hemodynamic sensor signal comprises an analog signal and the system comprises an analog-to-digital converter that converts the hemodynamic sensor signal to the arterial pressure signal waveform; and
determining the endotype of hypotension of the patient includes converting, using the analog-to-digital converter, the hemodynamic sensor signal to the arterial pressure signal waveform.

13. The hemodynamic sensor system of claim 1, wherein determining the endotype of hypotension of the patient comprises:
extracting from the arterial pressure signal waveform a plurality of heart health parameters;
comparing the plurality of heart health parameters with a reference that describes a profile of each of the endotype clusters for each of the plurality of heart health parameters; and
determining the endotype of hypotension of the patient based on a best-fit model of the plurality of heart health parameters with the clusters.

14. The system of claim 13 wherein the clusters associated with the arterial pressure signal are formed in latent space and then used to generate physical parameters.

15. A hemodynamic sensor system configured to determine and display an endotype of hypotension of a patient, the system comprising:
a hemodynamic sensor that produces a hemodynamic sensor signal representative of an arterial pressure signal waveform of the patient;
a user interface;
a non-transitory memory having executable instructions stored thereon; and
an electronic hardware processor in communication with the non-transitory memory and configured to execute the instructions to cause the system to at least:
receive, from the hemodynamic sensor, the hemodynamic sensor signal from the patient;
determine, based on the arterial pressure signal waveform, the endotype of hypotension of the patient; and
generate, based on the determined endotype of hypotension of the patient, data for displaying on the user interface an alert indicating the endotype of hypotension of the patient.

16. The hemodynamic sensor system of claim 15, wherein determining the endotype of hypotension of the patient comprises:
extracting from the arterial pressure signal waveform a plurality of heart health parameters;
encoding, using a deep learning model, the plurality of heart health parameters into one or more latent space heart health parameters; and
generating a location in latent space of the arterial pressure signal waveform using the one or more latent space heart health parameters.

17. The hemodynamic sensor system of claim 16, wherein determining the endotype of hypotension of the patient further comprises:
determining, based on the location of the arterial pressure signal waveform in latent space, a relative location of the arterial pressure signal waveform in latent space based on a set of clusters from reference arterial pressure signal waveforms within the latent space;
determining, based on the set of clusters, a cluster associated with the arterial pressure signal waveform in latent space; and
based on the cluster of the arterial pressure signal waveform, determine the endotype of hypotension of the patient.

18. The hemodynamic sensor system of claim 16, wherein the alert includes a graphical user interface feature that indicates at least one of the plurality of heart health parameters that characterize the endotype of hypotension of the patient.

19. The hemodynamic sensor system of claim 18, wherein the at least one of the plurality of heart health parameters includes a stroke volume index (SVI), a heart rate (HR), a cardiac index (CI), a systemic vascular resistance index (SVRI), and a stroke volume variation (SVV).

20. The hemodynamic sensor system of claim 15, wherein the endotype of hypotension is selected from the following group: a vasodilation endotype, a myocardial depression endotype, a bradycardia endotype, and a hypovolemia endotype.

21. The hemodynamic sensor system of claim 15, wherein a plurality of heart health parameters are extracted from the arterial pressure signal waveform, wherein the heart health parameters are encoded into a latent space to form latent space heart health parameters, wherein generating data for display comprises causing presentation of the user interface, and wherein the user interface:
presents a plurality of clusters, wherein individual clusters are associated with individual endotypes, and wherein each cluster is formed from latent space heart health parameters associated with respective patients, and
updates based on information indicating a particular patient and presents an endotype trend associated with the particular patient.

22. The hemodynamic sensor system of claim 21, wherein one or more probability areas are included for each cluster, and wherein each probability area indicates a likelihood of a corresponding endotype associated with the cluster.

23. The hemodynamic sensor system of claim 15, wherein determining the endotype of hypotension of the patient comprises:
determining a hypotension probability index (HPI) corresponding to the probability of the patient developing the endotype of hypotension.

24. The hemodynamic sensor system of claim 23, wherein generating the data for displaying an alert indicating the endotype of hypotension of the patient is further based on the HPI being above a threshold HPI.

25. The hemodynamic sensor system of claim 15, wherein the processor is configured to execute the instructions to further cause the system to at least:
determine, based on the determined endotype of hypotension, a therapy protocol for the patient.

26. The hemodynamic sensor system of claim 25, wherein the processor is configured to execute the instructions to further cause the system to at least:
generate, based on the determined therapy protocol, a command configured to cause an infusion pump to deliver an intravenous therapeutic agent to the patient.

27. The hemodynamic sensor system of claim 26, further comprising the infusion pump.

28. The hemodynamic sensor system of claim 15, wherein determining the endotype of hypotension of the patient comprises:
extracting from the arterial pressure signal waveform a plurality of heart health parameters;
comparing the plurality of heart health parameters with a reference that describes a profile of each of the endotype clusters for each of the plurality of heart health parameters; and
determining the endotype of hypotension of the patient based on a best-fit model of the plurality of heart health parameters with the clusters.

29. The hemodynamic sensor system of claim 15, wherein:
the hemodynamic sensor signal comprises an analog signal and the system comprises an analog-to-digital converter that converts the hemodynamic sensor signal to the arterial pressure signal waveform; and
determining the endotype of hypotension of the patient includes converting, using the analog-to-digital converter, the hemodynamic sensor signal to the arterial pressure signal waveform.

30. A hemodynamic sensor system configured to determine an endotype of hypotension of a patient, the system comprising:
a hemodynamic sensor that produces an analog hemodynamic sensor signal representative of an arterial pressure signal waveform of the patient;
an analog-to-digital converter that converts the analog hemodynamic sensor signal to the arterial pressure signal waveform;
a non-transitory memory having executable instructions and a fully connected deep learning model stored thereon; and
an electronic hardware processor in communication with the non-transitory memory and configured to execute the instructions to cause the system to at least:
receive, from the hemodynamic sensor, the analog hemodynamic sensor signal from the patient;
convert, using the analog-to-digital converter, the analog hemodynamic sensor signal to the arterial pressure signal waveform;
extract from the arterial pressure signal waveform a plurality of heart health parameters;
encode, using the fully connected deep learning model, the plurality of heart health parameters into one or more latent space heart health parameters;
generate a location in latent space of the arterial pressure signal waveform using the one or more latent space heart health parameters;
determine, based on the location of the arterial pressure signal waveform in latent space, a relative location of the arterial pressure signal waveform in latent space, wherein determining the relative location of the arterial pressure signal waveform in latent space comprises:
obtaining a plurality of reference arterial pressure signal waveforms from a plurality of patients;
extracting from the plurality of reference arterial pressure signal waveforms a plurality of reference sets of heart health parameters;
combining each of the plurality of reference sets of heart health parameters into a plurality of corresponding one or more reference latent space heart health parameters;
generating reference locations in latent space of each of the plurality of reference arterial pressure signal waveforms based on the plurality of one or more reference latent space heart health parameters of each of the plurality of reference sets of heart health;
determining a clustering evaluation metric of the reference locations in latent space, the clustering evaluation metric configured to indicate a goodness of clustering of the reference locations;
based on the clustering evaluation metric, determining a best number of clusters associated with the reference locations;
associating, based on the best number of clusters, each of the reference arterial pressure signal waveforms to a corresponding cluster in latent space to determine a set of clusters; and
determining, based on the set of clusters, a cluster associated with the arterial pressure signal waveform in latent space;
based on the cluster of the arterial pressure signal waveform, determine the endotype of hypotension of the patient; and
generate, based on the determined endotype of hypotension of the patient, data for displaying an alert indicating the endotype of hypotension of the patient.
